(19) United States
(12) Patent Application Publication
(10) Pub. No.: US 2024/0424027 A1
(43) Pub. Date: Dec. 26, 2024

(54) Anti-Aging Composition Using Mesenchymal Stem Cells and Methods Thereof

(71) Applicant: Mechanogenic K.K., Media, PA (US)

(72) Inventor: Makoto Funaki, Media, PA (US)

(73) Assignee: Mechanogenic K.K., Media, PA (US)

(21) Appl. No.: 18/700,914

(22) PCT Filed: Oct. 12, 2022
(86) PCT No.: PCT/US2022/046461

§ 371 Date: Apr. 12, 2024

Related U.S. Application Data
(60) Provisional application No. 63/254,884, filed on Oct. 12, 2021.
(60) Provisional application No. 63/336,211, filed on Apr. 28, 2022.

(51) Int. Cl. A61K 35/28; A61K 8/98; A61P 11/00; A61P 17/02; A61P 29/00; A61P 37/02; C12N 5/0775

(52) U.S. Cl. CPC A61K 35/28; A61K 8/981; A61P 11/00; A61P 17/02; A61P 29/00; A61P 37/02; C12N 5/0663; C12N 5/0667

(57) ABSTRACT

Methods and compositions for reducing oxidative stress in a cell and/or decreasing aging or treating an aging-associated disease in a subject are provided. The methods and compositions include the use of quiescent mesenchymal stem cell (MSC) either alone or with a substrate that adheres to the quiescent MSC.

### FIELD OF THE INVENTION

This present invention relates to methods and compositions that use quiescent mesenchymal stem cells.

### BACKGROUND OF THE INVENTION

Mesenchymal stem cells (MSCs) are one type of somatic stem cells, which possess a capability of multilineage differentiation and immunomodulatory functions and are drawing much attention in regenerative medicine and cell therapies. MSC-derived secretome and extracellular vesicles have been attributable to their immunomodulatory functions to suppress immune reactions and inflammation in a disease state. Thus, multiple clinical trials have been attempted on MSCs. However, the feasibility of MSC-based cell therapies is far from being established due to either its insufficient efficacy and/or safety concerns.

In age-related diseases, declined immunomodulatory functions and anti-inflammatory functions of MSCs have been observed, which is caused by aging of MSCs, termed ‘senescence.’ Senescence refers to cell cycle arrest without immediate cell death, and cells keep metabolism but release harmful substance to cause inflammation and damages to other cells. MSCs cultured ex vivo for a prolonged period go through excess numbers of cell cycles and show permanent and irreversible cell cycle arrest due to an increased shortening of telomeres and increased expression of cell cycle-dependent kinase inhibitors. Immunomodulatory functions and anti-inflammatory functions in these replicative senescent cells are also diminished. In contrast, MSCs in age-related diseases and conditions are not necessarily replicative senescent. Rather, cellular stresses, such as oxidative stress, causing age-related diseases and conditions render MSCs ‘premature’ senescent state before they become replicative senescent. Senescent MSCs, whether replicative or premature, are known to exhibit a distinctive phenotype in their secretion profile of cytokines and extracellular vesicles, which is called senescence-associated secretary phenotype (SASP). SASP of senescent MSCs spread senescent phenotype to their neighboring cells and causes chronic inflammatory environment, which is understood as one of the underlying mechanism of some age-related diseases and conditions, such as cardiovascular diseases, chronic obstructive pulmonary disease, chronic kidney disease, diabetes and its complications, neurodegenerative diseases, cancers, autoimmune diseases, sarcopenia and frailty. Thus, treatment of MSC's SASP may lead to rejuvenation of affected tissues and organs, and even individuals, which includes disease fighting and regeneration implications.

### SUMMARY OF THE INVENTION

One aspect of the present invention is directed to a method of reducing oxidative stress in a cell.

Another aspect of the present invention is directed to a method of decreasing aging or treating an aging-associated disease in a subject. The method includes administering, to the subject, a composition comprising a quiescent mesenchymal stem cell (MSC) and a substrate that adheres to the quiescent MSC.

Another aspect of the present invention is directed to a composition that includes a quiescent mesenchymal stem cell (MSC) and a substrate that adheres to the quiescent MSC. In one embodiment, the MSC is adipose tissue-derived stromal cells (ADSCs).

Another aspect of the present invention is directed to a pharmaceutical composition configured for injection. The composition includes a quiescent mesenchymal stem cell (MSC) and a substrate that adheres to the quiescent MSC.

These and other embodiments, features and advantages of this invention will be more readily understood by those of ordinary skill in the art from a reading of the following detailed description and appended claims.

### BRIEF DESCRIPTION OF THE DRAWINGS

FIG. 1 depicts that ADSCs became quiescent, when cultured in 3D soft biocompatible gels.

FIG. 2 depicts that IDO expression in an inflammatory condition was drastically upregulated in quiescent ADSCs, when compared with the level of upregulation observed in non-quiescent ADSCs.

FIG. 3 depicts that IL-6 secretion was attenuated by oxidative stress in non-quiescent ADSCs, but kept intact in ADSCs, which were exposed to oxidative stress while they were non-quiescent but became quiescent thereafter.

FIG. 4 depicts that MCP-1 secretion was enhanced by oxidative stress in non-quiescent ADSCs, but was abrogated in ADSCs exposed to oxidative stress while they were non-quiescent but became quiescent thereafter.

FIG. 5 depicts that the viability of HUVEC was enhanced by conditioned medium from ADSCs even under oxidative stress.

FIGS. 6A and 6B depict that Quiescent ADSCs in 3D-NANOFIBGROW-I gels intraperitoneally administrated into mice were detected 7 days later. 10⁶ ADSCs were embedded in a 300 μl 3D-NANOFIBGROW-I gel and intraperitoneally injected into mice. 7 days after ADSC administration, mice were sacrificed. Three mice were used for the experiment and representative images are shown. In FIG. 6A, gels were identified in the abdominal cavity (arrowheads). In FIG. 6B, gels identified in the abdominal cavity were isolated and stained with hematoxylin and eosin. The right panel is a higher-magnification image of the boxed area in the left panel. In FIG. 6C, 300 μl 3D-NANOFIBGROW-I gels containing either vehicle (PBS) or 3×10⁶ ADSCs were prepared. Either a PBS-containing gel or an ADSC-containing gel was intraperitoneally injected into a mouse, respectively. Either vehicle or LPS (5 mg/kg) was intraperitoneally injected into mice at the same time of 3D-NANOFIBGROW-I gel injection. Seven days after injection, mice were sacrificed and lungs were isolated for hematoxylin and eosin staining. Left panel; vehicle (PBS)+vehicle (normal saline), Middle Panel; vehicle (PBS)+LPS, Right panel; ADSCs+LPS. Six mice were used for each group and representative images are shown.

FIG. 7 depicts that a pro-inflammatory environment in vitro did not affect proliferation of MSCs.

MSCs were sparsely seeded on either 250 Pa or 7500 Pa polyacrylamide gels. Single cells on each gel were marked on the next day and they were treated with either vehicle or a combination of 20 ng/ml TNFα and 20 ng/ml IFNγ. Four days later the number of cells in each marked area was counted. Representative images (A) and quantitative results (3 areas per each condition) are shown (B). Data are expressed as means±S.D. Similar results were obtained in two other independent experiments.

FIG. 8. Production of immunomodulatory factors by quiescent MSCs in a pro-inflammatory environment. MSCs were seeded on 250 Pa polyacrylamide gels and treated with a combination of 20 ng/ml TNFα and 20 ng/ml IFNγ for the indicated time. Cells were then lysed and lysates were immunoblotted with anti-HGF (top panel), anti-IDO (middle panel) or anti-α-tubulin antibodies (bottom panel).

FIG. 9. Inflammation in the lacrimal gland of Sjöegren's syndrome model mice was significantly suppressed by intraperitoneal administration of MSCs embedded in soft biocompatible and injectable gel. Sjöegren's syndrome model mice were prepared as described previously (Ishimaru N, Saegusa K, Yanagi K, Haneji N, Saito I, et al. (1999) Estrogen deficiency accelerates autoimmune exocrinopathy in murine Sjögren's syndrome through Fas-mediated apoptosis. Am J Pathol 155: 173-181). Either VitroGel RGD-PLUS only (Gel) or a mixture of MSCs and VitroGel RGD-PLUS (MSC/Gel) were intraperitoneally injected into those mice, when they reached 7 weeks old and once a week thereafter until they reached 12 weeks old. Then mice were sacrificed and their lacrimal glands and salivary glands were isolated. Histopathological grading was conducted and the number of infiltrated lymphocytes was counted after hematoxylin and eosin staining as described previously (Ishimaru N et al.). In FIG. 9A, representative histology images of lacrimal glands (LG) and salivary glands (SG). In FIG. 9B-C, histopathological grading (B) and the number of lymphocytes (C) in mice are shown. 5 mice were used for each group. Data are expressed as means±standard deviation of triplicates for each group.

FIG. 10. The populations of CD4⁺ CD44^{high} CD62L⁻ T cells in both the cervical lymph nodes and spleen were significantly decreased in Sjöegren's syndrome model mice by intraperitoneal administration of a mixture of MSCs and a soft biocompatible and injectable gel. Sjöegren's syndrome model mice were prepared and treated with either VitroGel RGD-PLUS only (Gel) or a mixture of MSCs and VitroGel RGD-PLUS (MSC/Gel). When mice reached 12 weeks old, they were sacrificed and their cervical lymph nodes (cLN) and spleens (Sp) were isolated. The percentage of CD62L(low)CD44(high) memory phenotype CD4(+) T cells in their cervical lymphnodes and spleens was analyzed by flow cytometry. A. Representative dot blot images are shown. B. Data are expressed as means±standard deviation obtained from 5 mice for each group. ***p<0.01, *p<0.05

FIG. 11. The populations of CD4⁺ PD-1⁺ CXCR5⁺ Foxop3⁻ cells in the cervical lymph nodes and spleen were significantly decreased in Sjöegren's syndrome model mice by intraperitoneal administration of a mixture of MSCs and soft biocompatible and injectable gels. Sjöegren's syndrome model mice were prepared and treated with either VitroGel RGD-PLUS only (Gel) or a mixture of MSCs and VitroGel RGD-PLUS (MSC/Gel) as described in FIG. 10. When mice reached 12 weeks old, they were sacrificed and their cervical lymph nodes (cLN) and spleens (Sp) were isolated. The percentage of percentage of PD-1(high)CXCR5(high) follicular helper CD4(+) T cells in their cervical lymphnodes and spleens was analyzed by a flow cytometry. A. Representative dot blot images are shown. B. Data are expressed as means±standard deviation obtained from 5 mice for each group. ***p<0.01, *p<0.05.

FIG. 12 depicts that BMSCs induced and maintained in quiescence by culturing them on biocompatible gels became non-quiescent upon contact with glass on top of them.

FIG. 13 depicts that ADSCs cultured in 3D in 3D-NANOFIBGROW-I gels exhibited cell cycle arrest.

FIG. 14 depicts that attenuation of dehydrogenase activity in quiescent ADSCs was reversible.

FIG. 15 depicts that quiescent ADSCs were resistant to high glucose-induced reduction in dehydrogenase activity.

FIG. 16 and FIG. 17 depict that DiI-labeled quiescent BMSCs (FIG. 16) and ADSCs (FIG. 17) were detected as round cells in gels even after 30 days of subcutaneous injection.

FIG. 18 depict that transplanted MSCs in gels remained as MSCs and enhanced angiogenesis in vivo.

FIG. 19 depicts that angiogenesis is enhanced in gels containing quiescent ADSCs.

FIG. 20 depicts that quiescent ADSCs are still found in gels even 72 days after subcutaneous transplantation.

FIG. 21 depicts that transplanting quiescent ADSCs in gels accelerates wound healing in diabetic mice.

FIG. 22 depicts that ADSCs exhibiting SASP can be rejuvenated by making them quiescent and accelerate wound healing in diabetic mice.

### DETAILED DESCRIPTION OF THE INVENTION

Before the present methods and compositions are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by appended claims.

In the context of the present description, all publications, patent applications, patents and other references mentioned herein, if not otherwise indicated, are explicitly incorporated by reference herein in their entirety for all purposes as if fully set forth.

Unless otherwise defined, all technical and scientific terms used herein have the same meaning as commonly understood by one of ordinary skill in the art to which this disclosure belongs. In case of conflict, the present specification, including definitions, will control.

Unless stated otherwise, all percentages, parts, ratios, etc., are by weight.

In certain instances, a quantitative value set forth herein may be determined by an analytical or other measurement method that is defined by reference to a published or otherwise recognized standard procedure. Typical examples of sources of such recognized standard procedures include ASTM (American Society for Testing Materials, now ASTM International); ISO (International Organization for Standardization); DIN (Deutsches Institut für Normung); and JIS (Japanese Industrial Standards). Unless clearly stated otherwise herein, the specific standard procedure used herein is considered to be the version of that procedure that is in force on the filing date of this application.

When an amount, concentration, or other value or parameter is given as a range, or a list of upper and lower values, this is to be understood as specifically disclosing all ranges formed from any pair of any upper and lower range limits, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

When a range of values is stated as being “less than” or “no more than” a designated quantity (or other equivalent phrasing), it is to be understood that the range is bounded on the low end by an unspecified non-zero value. Correspondingly, when a range of values is stated as being “more than”, “greater than”, or “not less than” a designated quantity (or other equivalent phrasing), it is to be understood that the range on the high end is not infinite, and that it is bounded on the high end by an unspecified finite value.

When the term “about” or “approximately” is used in describing a value or an end-point of a range, the disclosure should be understood to include the specific value or end-point referred to. Whether modified by the term “about” the claims appended hereto include equivalents to these quantities. The term “about” further may refer to a range of values that are similar to the stated reference value. In certain embodiments, the term “about” refers to a range of values that fall within 50, 25, 10, 9, 8, 7, 6, 5,4, 3, 2, 1 percent or less of the stated reference value.

As used herein, the terms “comprises,” “comprising,” “includes,” “including,” “has,” “having” or any other variation thereof, are intended to cover a non-exclusive inclusion. For example, a process, method, article, or apparatus that comprises a list of claim elements is not necessarily limited to only those elements but can include other elements not expressly listed or inherent to such process, method, article, or apparatus.

The transitional phrase “consisting of” excludes any claim element or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified claim elements, materials or steps and those others that do not materially affect the basic and novel characteristic(s) of the claimed invention. A “consisting essentially of” claim thus occupies a middle ground between closed claims that are written in a “consisting of” format, and fully open claims that are drafted in a “comprising” format. Optional additives as defined herein, at a level that is appropriate for such additives, and minor impurities are not excluded from a composition by the term “consisting essentially of”.

Further, unless expressly stated to the contrary, “or” and “and/or” refers to an inclusive and not to an exclusive. For example, a condition A or B, or A and/or B, is satisfied by any one of the following: A is true (or present) and B is false (or not present), A is false (or not present) and B is true (or present), and both A and B are true (or present).

The use of “a” or “an” to describe the various elements and components herein is merely for convenience and to give a general sense of the disclosure. This description should be read to include one or at least one and the singular also includes the plural unless it is obvious that it is meant otherwise.

In one aspect, the present disclosure relates to a method of reducing oxidative stress in a cell, comprising contacting the cell with a quiescent mesenchymal stem cell (MSC). “Contact” as used herein refers to direct cell-to-cell contact as well as indirect contact through secreted signaling molecules and structures, such as amino acids, proteins, lipids, nucleic acids (e.g., mRNAs), enzymes, hormones, neurotransmitters, ectosomes, and exosomes, or placing a target cell in a microenvironment or niche where a MSC resides. “Mesenchymal” stem cells of methods and compositions of the present disclosure are isolated or purified, in another embodiment, from bone marrow. In another embodiment, the cells are bone marrow-derived mesenchymal stem cell. In another embodiment, the cells are isolated or purified from adipose tissue. In some embodiments, the MSC is adipose tissue-derived stromal cells (ADSCs). In some embodiments, a source of the MSC may be an umbilical cord. In some embodiments, a source of the MSC may be dental pulp. In some embodiments, a source of the MSC may be Wharton's jelly. In some embodiments, a source of the MSC may be amniotic fluid. In some embodiments, a source of the MSC may be placenta. In some embodiments, a source of the MSC may be peripheral blood. In some embodiments, a source of the MSC may be synovium. In some embodiments, a source of the MSC may be synovial fluid. In some embodiments, a source of the MSC may be endometrium. In some embodiments, a source of the MSC may be a dermal tissue. In some embodiments, a source of the MSC may be skin. In some embodiments, a source of the MSC may be muscle.

In another embodiment, the cells are isolated or purified from cartilage. In another embodiment, the cells are isolated or purified from any other tissue known in the art. Each possibility represents a separate embodiment of the present invention.

Definitions of “quiescent” can include but are not limited to the following. “Quiescent” refers to a lack of significant replication. In another embodiment, the term refers to a significantly reduced level of replication. In another embodiment, the term refers to a large percentage of cells arrested in the cell cycle. In another embodiment, the cells are arrested at the G1 phase. In another embodiment, the cells are arrested in the G2 phase. In another embodiment, “quiescent” refers to any other art-accepted definition of the term. Each possibility represents a separate embodiment of the present invention. In some embodiments, “quiescent” MSCs do not exhibit SASP. In some embodiments, the quiescent MSCs described herein is characterized by (i) a lack of proliferation, (ii) a lack of differentiation, (iii) the ability of the cell to express proteins, and/or (iv) the ability of the cell to resume proliferation and differentiation upon exposure to a chemical stimulus, a mechanical stimulus, a physical factor or a combination thereof.

Methods for determining proliferative capacity and differentiation potency of mesenchymal stem cells are well known in the art, and are described, for example, in Baxter MA et al (Study of telomere length reveals rapid aging of human marrow stromal cells following in vitro expansion Stem Cells 2004, 22(5) 675-82), Liu L et al (Telomerase deficiency impairs differentiation of mesenchymal stem cells Exp Cell Res 2004 Mar. 10, 294(1) 1-8), and Bonab M M et al (Aging of mesenchymal stem cell in vitro BMC Cell Biol 2006 Mar. 10, 7 14). Each possibility represents another embodiment of the present invention.

In some embodiments, the cell contacted by the quiescent MSC is an organ cell. In some embodiments, the cell is an endothelial cell. In additional embodiments, the cell is a lung cell. In additional embodiments, the cell is a skin cell. In additional embodiments, the cell may be one or more cells selected from the group consisting of cardiomyocyte, endothelial cell, vascular smooth muscle cell, fibroblast, and myofibroblast. In additional embodiments, the cell may be one or more cells selected from the group consisting of macrophage, monocyte, dendritic cell, and immune cell. In additional embodiments, the cell may be one or more cells selected from the group consisting of lung epithelial cell and bronchial epithelial cell. In additional embodiments, the cell may be one or more cells selected from the group consisting of tubular epithelial cell, podocyte, interstitial cell, and mesangial cell. In additional embodiments, the cell may be one or more cells selected from the group consisting of adipocyte, myotube, myocyte, hepatocyte, biliary epithelial cell, and pancreatic beta cell. In additional embodiments, the cell may be one or more cells selected from the group consisting of retinal cell, neuronal cell, and glial cells. In additional embodiments, the cell may be a cancer cell. In additional embodiments, the cell may be one or more cells selected from the group consisting of keratinocyte and melanocyte. In additional embodiments, the cell may be one or more cells selected from the group consisting of gastrointestinal epithelial cell and colon epithelial cell. In additional embodiments, the cell may be one or more cells selected from the group consisting of osteoblast, osteocyte, and osteoclast. In additional embodiments, the cell may be a gland cell. In additional embodiments, the cell may be one or more cells selected from the group consisting of hematopoietic stem cell and progenitor cell.

In some embodiments, the quiescent MSC is prepared by culturing an MSC on a substrate having a rigidity from about 150 Pa to about 750 Pa. In additional embodiments, the quiescent MSC is prepared by culturing an MSC on a substrate having a uniform rigidity from about 150 Pa to about 750 Pa. In further embodiments, the substrate is a gel. In yet further embodiments, the gel may be 2-dimensional gel or 3-dimensional gel.

The culturing may be performed, in another embodiment, for at least 5 days. In another embodiment, the step of culturing is performed for at least 4 days. In another embodiment, the step of culturing is performed for at least 6 days. In another embodiment, the step of culturing is performed for at least 7 days. In another embodiment, the step of culturing is performed for at least 8 days. In another embodiment, the step of culturing is performed for at least 10 days. In another embodiment, the step of culturing is performed for at least 12 days. In another embodiment, the step of culturing is performed for at least 15 days. In another embodiment, the step of culturing is performed for at least 20 days. In another embodiment, the step of culturing is performed for at least 25 days. In another embodiment, the step of culturing is performed for at least 30 days. In another embodiment, the step of culturing is performed for at least 35 days. In another embodiment, the step of culturing is performed for at least 40 days. In another embodiment, the step of culturing is performed for at least 50 days. In another embodiment, the step of culturing is performed for at least 60 days. In another embodiment, the step of culturing is performed for over 4 days. In another embodiment, the step of culturing is performed for over 6 days. In another embodiment, the step of culturing is performed for over 7 days. In another embodiment, the step of culturing is performed for over 8 days. In another embodiment, the step of culturing is performed for over 10 days. In another embodiment, the step of culturing is performed for over 12 days. In another embodiment, the step of culturing is performed for over 15 days. In another embodiment, the step of culturing is performed for over 20 days. In another embodiment, the step of culturing is performed for over 25 days. In another embodiment, the step of culturing is performed for over 30 days. In another embodiment, the step of culturing is performed for over 35 days. In another embodiment, the step of culturing is performed for over 40 days. In another embodiment, the step of culturing is performed for over 50 days. In another embodiment, the step of culturing is performed for over 60 days. In another embodiment, the step of culturing is performed for 4 days. In another embodiment, the step of culturing is performed for 6 days. In another embodiment, the step of culturing is performed for 7 days. In another embodiment, the step of culturing is performed for 8 days. In another embodiment, the step of culturing is performed for 10 days. In another embodiment, the step of culturing is performed for 12 days. In another embodiment, the step of culturing is performed for 15 days. In another embodiment, the step of culturing is performed for 20 days. In another embodiment, the step of culturing is performed for 25 days. In another embodiment, the step of culturing is performed for 30 days. In another embodiment, the step of culturing is performed for 35 days. In another embodiment, the step of culturing is performed for 40 days. In another embodiment, the step of culturing is performed for 50 days. In another embodiment, the step of culturing is performed for 60 days. In another embodiment, the step of culturing is performed for over 60 days. In another embodiment, the step of culturing the mesenchymal stem cell population in a gel or matrix of the present invention is preceded by a step of culturing the mesenchymal stem cells in a tissue culture apparatus. In another embodiment, the tissue culture apparatus is a dish. In another embodiment, the tissue culture apparatus is a plate. In another embodiment, the tissue culture apparatus is a flask. In another embodiment, the tissue culture apparatus is a bottle. In another embodiment, the tissue culture apparatus is a tube. In another embodiment, the tissue culture apparatus is any other type of tissue culture apparatus known in the art, including those capable of culturing 3-dimensional spheroids. In another embodiment, the step of culturing is preceded by a step of culturing the mesenchymal stem cells in tissue-culture media; e.g. not in the presence of a gel or matrix of the present invention. In another embodiment, the step of culturing the cells in a tissue culture apparatus or in tissue culture media is performed after isolation of the mesenchymal stem cell population from a biological sample. In another embodiment, the step of culturing is performed after purification of the mesenchymal stem cell population from a biological sample. In another embodiment, the step of culturing is performed after enrichment of the mesenchymal stem cell population in a biological sample. Each possibility represents a separate embodiment of the present invention.

In some embodiments, soft-gels, which have optimized viscoelastic properties, may be used to produce quiescent MSCs. In some embodiments, exemplary methods to produce quiescent cells are disclosed in U.S. Pat. Nos. 10,214,720 and 11,083,190, which are incorporated by reference herein in their entirety.

In one embodiment, the gel matrix described herein are capable of forming gels of various strength, depending on their structure and concentration as well as, in another embodiment, environmental factors such as ionic strength, pH and temperature. The combined viscosity and gel behavior referred to as “viscoelasticity” in one embodiment, are examined by determining the effect that an oscillating force has on the movement of the material. In another embodiment elastic modulus (G′), viscous modulus (G″), and complex viscosity (η*) are the parameters sought to be changed using the methods described herein, and these are analyzed in another embodiment by varying either stress or strain harmonically with time. These parameters are derived from the complex modulus (G*), which is the ratio of maximum stress to maximum strain, and the phase angle (ω), which is the angle that the stress and strain are out of phase.

In one embodiment, in the gel matrices described herein, some of the deformation caused by shear stress is elastic and will return to zero when the force is removed. The remaining deformation such as that deformation created by the sliding displacement of the chains through the solvent in one embodiment will not return to zero when the force is removed. Under a constant force the elastic displacement remains constant in one embodiment, whereas the sliding displacement continues, so increasing.

In one embodiment, the term “elastic,” or “elasticity,” and like terms refer to a physical property of the gel matrices described herein, namely the deformability of the gel under mechanical force and the ability of the gel matrix to retain its original shape when the deforming force is removed. In another embodiment, the term “elastic modulus” refers to Young's Modulus and is a measure of the ratio of (a) the uniaxial stress along an axis of the material to (b) the accompanying normal strain along that axis.

The shear modulus (resulting from changing strain) is the ratio of the shear stress to the shear strain. It follows from the complex relationship similar to the above that:

$$G *= {G}^{′} + {iG}^{″}$$

where G* is the complex shear modulus, G′ is the in-phase storage modulus, i is a material-related factor and G″ is the out-of-phase similarly-directed loss modulus; G*=E(G′2+G″2). The frequency where these parameters cross over corresponds to a relaxation time (τ) specific for the material.

In one embodiment, linear viscoelastic properties of the gel matrices described herein are determined by measurements in an oscillating shear flow at small amplitude and with variable angular frequency. The values for G′ and G″ are determined to a great extent here by the concentration of a polymer in the aqueous solution and the magnitude of the representative viscosity value. Therefore, hereinafter, only the relative course of G′ and G″ with increasing angular frequency ω, is considered. In another embodiment, at a concentration of 1.5 to 2% (w/w) of a polymer of aqueous solution and a temperature of approximately 20° C., the behavior of G′ and G″ for the a polymer is such that at a low angular frequency (ω, the storage modulus G′ is less than the loss modulus G″, but with increasing angular frequency G′ increases more greatly than G″. In another embodiment, G′, above a certain angular frequency, finally becomes greater than G″, and the solution at high values of angular frequency thus predominantly reacts elastically. This behavior is attenuated or changed using the modulating methods described herein.

In one embodiment, rigidity or stiffness, refers to the G′ values observed or measured.

In another embodiment, a substrate, a gel or a gel matrix described herein may be coated with a solution comprising an adhesion protein. In another embodiment, the adhesion protein is a collagen. In another embodiment, the adhesion protein is a type 1 collagen. In another embodiment, the adhesion protein is a fibronectin. In another embodiment, the adhesion protein is any other adhesion protein known in the art. In another embodiment, the gel or matrix is coating with a solution comprising a combination of adhesion proteins. In another embodiment, the gel or matrix is coating with a solution comprising a collagen and a fibronectin. In another embodiment, the gel or matrix is coating with a solution comprising a type I collagen and a fibronectin. Each possibility represents a separate embodiment of the present disclosure. In some embodiments, the gel or matrix described herein may include an adhesion molecule. The “adhesion molecule” refers to a molecule capable of mediating adhesion between a substrate and a cell, such as an MSC described herein. In additional embodiment, the adhesion molecule may be a peptide comprising RGD. In additional embodiment, the adhesion molecule may be a peptide comprising an integrin molecule.

In another embodiment, the collagen of methods and compositions of the present disclosure is a recombinant collagen. In another embodiment, the collagen is purified from a biological source. In another embodiment, the collagen is a type 1 collagen. In another embodiment, the collagen is any other type of collagen known in the art. Each possibility represents a separate embodiment of the present disclosure.

In another embodiment, the fibronectin of methods and compositions of the present disclosure is a recombinant fibronectin. In another embodiment, the fibronectin is purified from a biological source. In another embodiment, the fibronectin is a type 1 fibronectin. In another embodiment, the fibronectin is any other type of fibronectin known in the art. Each possibility represents a separate embodiment of the present disclosure.

In some embodiments, the gel described herein comprises a gelling agent. The gelling agent of methods and compositions of the present disclosure is, in another embodiment, an acrylamide. In another embodiment, the gelling agent is an acrylamide-bisacrylamide mixture. In another embodiment, the gelling agent comprises acrylamide. In another embodiment, the gelling agent comprises an acrylamide-bisacrylamide mixture. Each possibility represents a separate embodiment of the present disclosure.

In some embodiments, the gel described herein comprises a nanofiber. In some embodiments, the gel described herein excludes a covalently bound polymer.

In one embodiment, a gel matrix having a rigidity in a range of 150-750 Pa; and an adipocyte induction medium, wherein said gel or matrix is coated with a type 1 collagen, a fibronectin, or a combination thereof are provided. In another embodiment, the gel matrix comprises a gelling agent and an acrylamide-bisacrylamide mixture. In some embodiments, said gel matrix is coated or comprises with a type 1 collagen, a fibronectin, or a combination thereof and having a rigidity in a range of 150-750 Pa. In another embodiment, the gel matrix comprises a gelling agent wherein said gel matrix is coated with a type 1 collagen, a fibronectin, or a combination thereof and wherein said gel matrix is maintained at a predetermined rigidity; and exposing the gel matrix to a growth modulating factor. In another embodiment, the gel matrix comprises an extracellular material that binds to integrin on the membrane of the somatic stem cell, said gel matrix having a substantially similar elasticity to the elasticity of the predominant in vivo biological microenvironment of the somatic stem cell of the same type in vivo; and providing the somatic stem cell with nutrient material for sustaining biological activity of the somatic stem cell ex vivo. In some embodiments, the gel matrix may have a rigidity at least of 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, or 740 Pa. In some embodiments, the gel matrix may have a rigidity of 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750 Pa or less.

Protease inhibitors can be included with the gel or gel matrix. The protease inhibitor can be a protein. In some embodiments, the protease inhibitor is a cysteine protease inhibitor, a serine protease inhibitor (serpin), a trypsin inhibitor, a threonine protease inhibitor, an aspartic protease inhibitor, or a metallo-protease inhibitor. In other embodiments, a protease inhibitor is a suicide inhibitor, a transition state inhibitor, or a chelating agent. The protease inhibitor can be a soybean trypsin inhibitor (SBTI). In another embodiment, the protease inhibitor is AEBSF-HCl. In another embodiment, the inhibitor is (epsilon)-aminocaproic acid. In another embodiment, the inhibitor is (alpha) 1-antichymotypsin. In another embodiment, the inhibitor is antithrombin III. In another embodiment, the inhibitor is (alpha) 1-antitrypsin ([alpha]1-proteinase inhibitor). In another embodiment, the inhibitor is APMSF-HCl (4-amidinophenyl-methane sulfonyl-fluoride). In another embodiment, the inhibitor is aprotinin. In another embodiment, the inhibitor is benzamidine-HCl. In another embodiment, the inhibitor is chymostatin. In another embodiment, the inhibitor is DFP (diisopropylfluoro-phosphate). In another embodiment, the inhibitor is leupeptin. In another embodiment, the inhibitor is PEFABLOC® SC (4-(2-Aminoethyl)-benzenesulfonyl fluoride hydrochloride). In another embodiment, the inhibitor is PMSF (phenylmethyl sulfonyl fluoride). In another embodiment, the inhibitor is TLCK (1-Chloro-3-tosylamido-7-amino-2-heptanone HCl). In another embodiment, the inhibitor is TPCK (1-Chloro-3-tosylamido-4-phenyl-2-butanone). In another embodiment, the inhibitor is trypsin inhibitor from egg white (Ovomucoid). In another embodiment, the inhibitor is trypsin inhibitor from soybean. In another embodiment, the inhibitor is aprotinin. In another embodiment, the inhibitor is pentamidine isethionate. In another embodiment, the inhibitor is pepstatin. In another embodiment, the inhibitor is guanidium. In another embodiment, the inhibitor is alpha2-macroglobulin. In another embodiment, the inhibitor is a chelating agent of zinc. In another embodiment, the inhibitor is iodoacetate. In another embodiment, the inhibitor is zinc. Each possibility represents a separate embodiment of the present disclosure.

Recombinant fibrin or fibrinogen protein can be included as a gelling agent. The fibrin or fibrinogen protein can be of a heterothermic animal. In another embodiment, the fibrin or fibrinogen protein is a fibrin or fibrinogen protein of a homeothermic animal. In another embodiment, the fibrin or fibrinogen is from a fish. In another embodiment, the fibrin or fibrinogen is from a salmon. In another embodiment, the fibrin or fibrinogen is from any other fish known in the art. In another embodiment, the fibrin or fibrinogen is from any other heterothermic known in the art. In another embodiment, the fibrin or fibrinogen is from a mammal. In another embodiment, the fibrin or fibrinogen is human fibrin or fibrinogen. In another embodiment, the fibrin or fibrinogen is bovine fibrin or fibrinogen. In another embodiment, the fibrin or fibrinogen is from any other mammal known in the art. In another embodiment, the fibrin or fibrinogen is from any other homoeothermic known in the art. Each possibility represents a separate embodiment of the present disclosure.

In one aspect, the present disclosure relates to preventing, ameliorating, treating an aging-associated condition in a subject, comprising administering an effective amount of a composition comprising a quiescent MSC described above and a substrate that adheres to the quiescent MSC to the subject. In some embodiments, the substrate may be a gel or gel matrix.

In some embodiments, the substrate or gel administered to the subject may or may not be the same substrate or gel on or in which the MSC is previously cultured to maintain or induce the quiescent state. The substrate that adheres to the quiescent MSC may be the gel to maintain and induce the quiescent state in MSC as described above.

In some embodiments, the aging-associated condition is caused by oxidative stress. In additional embodiments, the oxidative stress is a chronic oxidative stress.

In some embodiments, the aging-associated condition is sarcopenia. In some embodiments, the aging-associate condition is frailty. In some embodiments, the aging-associate condition is an aging-associated disease. In some embodiments, the aging-associated condition is a decreased wound healing. In some embodiments, the aging-associated condition is a decreased wound healing in diabetes. In some embodiments, the aging-associated condition is a diabetic ulcer. In some embodiments, the aging-associated condition is an orthodontal disease. In some embodiments, the aging-associated condition is an autoimmune disease. In some embodiments, the aging-associated condition is an inflammatory disease. In some embodiments, the aging-associated condition is an inflammatory respiratory disease. In some embodiments, the aging-associated condition is an inflammatory respiratory disease caused by a virus. In some embodiments, the aging-associated condition is an inflammatory respiratory disease caused by a coronavirus. In some embodiments, the aging-associated condition is an acute lung injury. In some embodiments, the aging-associated condition is an LPS-induced acute lung injury. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, presence of pre-existing conditions other than age-associated diseases/conditions, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

In some embodiments, the methods of treating the disease provide a positive therapeutic response with respect to a disease or condition. By “positive therapeutic response” is intended an improvement in the disease or condition, and/or an improvement in the symptoms associated with the disease or condition. The therapeutic effects of the subject methods of treatment can be assessed using any suitable method. In some embodiments, the subject methods reduce the amount of a disease-associate protein deposition in the subject by at least 5%, 10%, 15%, 20%, 25%, 30%, 35%, 40%, 45%, 50%, 55%, 60%, 65%, 70%, 75%, 80%, 85%, 90%, 95%, or 99% as compared to the subject prior to undergoing treatment.

In some embodiments, the subjects that can be treated with the methods described herein include, but are not limited to, mammalian subjects such as a mouse, rat, dog, baboon, pig or human. In some embodiments, the subject is a human. The methods can be used to treat subjects at least 25 years, 30 years, 35 years, 40 years, 45 years, 50 years, 55 years, 60 years, 65 years, 70 years, 75 years, 80 years, 85 years, 90 years, 95 years or 100 years of age. In some embodiments, the subject is treated for at least one, two, three, or four diseases.

In some embodiments, the administering is performed by injection, micro-dermal injection, or topical application. In some embodiments, the administering is performed by intraperitoneal, subcutaneous, intramuscular or intravenous injection.

In some embodiments, the composition may be a cosmetic composition. In some embodiments, the composition may be a pharmaceutical composition.

In one aspect, the present disclosure relates to a cosmetic or pharmaceutical composition comprising a quiescent MSC and a substrate that adheres to the quiescent MSC as disclosed above. As discussed above, the substrate may be a gel or a gel matrix.

Pharmaceutical and cosmetic compositions may include carriers including, but not limited to, a diluent, adjuvant, excipient, or vehicle with which a hyaluronidase, with or without one or more additional Active Pharmaceutical Ingredients (“APIs”), or immunoglobulin (IG) is administered. Examples of suitable pharmaceutical carriers are described in “Remington's Pharmaceutical Sciences” by E. W. Martin. Such compositions may contain a therapeutically effective amount of the compound, generally in purified form or partially purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

The methods of the present invention may be practiced in vivo as well as ex vivo. Such systems may, for example, include porous structures for insertion in specific tissues or the circulatory system for maintaining a stem cell in quiescence within the body. For example, stem cells, corresponding ECM and, optionally, linking material, may be dispersed in a polymeric matrix that has appropriate elasticity apparent to the stem cells to induce or maintain quiescence, and that also has sufficient porosity to permit in vivo nutrients to reach the cell and to permit proteins and other factors expressed by the cell to leave the matrix. Other embodiments may include cassettes or other devices that induce or maintain quiescence in stem cells, and that may be implanted into a host.

A further aspect of the present invention encompasses using a quiescent stem cell sustained in biological activity ex vivo. Examples of a stem cell described herein may include a somatic stem cell or an embryonic stem cell, a human stem cell or an animal stem cell, a mesenchymal stem cell (MSC), a bone marrow-derived MSCs, a renal stem cell, a hepatic-derived stem cell, a skeletal muscle-derived MSC, a bone-derived MSC, a dental pulp MSC, a cardiac muscle-derived MSC a synovial-fluid derived MSC or an umbilical cord MSC.

Some aspects of the present disclosure related to reversing stress-induced senescence-associated secretion phenotype (SASP) in adipose tissue-derived stromal cells (ADSCs), one type of MSCs most frequently tested in search for MSC-based cell therapies due to their strong immunomodulatory and anti-inflammatory functions and easy access to the source by introducing and maintaining quiescence. Evidence is provided that oxidative stress, which is known to play a major role in age-related diseases and conditions, does cause premature senescence and SASP in non-quiescent ADSCs. However, introduction and maintenance of quiescence in those ADSCs by culturing them in soft environment may abrogate premature senescence of ADSCs and eliminates oxidative stress-induced death in their neighboring endothelial cells. In addition, evidence is provided that quiescent ADSCs may exert anti-inflammatory functions in vivo. Secretome and extracellular vesicles (exosomes) originated from senescent cells may affect neighboring or nearby cells. In addition, in present MSC-based cells therapies MSCs intravenously injected or locally administrate may migrate into inflamed damaged tissues and organs. On the other hand, the data supports the idea that inraperitoneally administrated ADSCs in gels stay in the peritoneum but are still able to suppress lung inflammation, which is remote from where ADSCs are.

Mesenchymal stem cells (MSCs) are one type of stem cells most promising in cell therapies for various diseases based on their immunomodulatory and anti-inflammatory functions. However, senescence of MSCs caused by cellular stresses abrogates their anti-inflammatory functions and, rather, propagate inflammation in neighboring cells and tissues through their secretion profile, called senescence-associated secretion phenotype (SASP). In some embodiments, the quiescence may reverse stress-induced senescence and that loss of SASP in quiescent MSCs would rejuvenate neighboring cells. To this end, adipose tissue-derived stromal cells (ADSCs) were placed in soft gels to make them quiescent. These ADSCs drastically upregulated indoleamine 2,3-dioxygenase (IDO) expression even in the presence of TNF a and IFNγ. Oxidative stress attenuated IL-6 secretion and stimulated MCP-1 by non-quiescent ADSCs, whereas it stimulated IL-6 secretion and attenuated MCP-1 secretion by ADSCs rendered quiescent after being exposed to oxidative stress while cells were still in non-quiescent state. These quiescent ADSCs successfully rescued endothelial cells from stress-induced cell death. Seven days after intraperitoneally administrating a mixture of gels and ADSCs into mice, ADSCs were still identified locally and successfully prevented LPS-induced acute lung injury in mice. These results suggest that quiescence can revert aging process of ADSCs and rejuvenate other cells locally and remotely.

### EXAMPLES

Abbreviations—TNF; Tumor necrosis factor, IFN; Interferon, 2D; two-dimensional, 3D; three-dimensional, Pa; pascal, MSC; mesenchymal stem cell, ADSC; adipose-derived stromal/stem cell, BMSC; bone marrow-derived stem cell, NFBC; Nanofiber bacterial cellulose, Pa; pascal, H&E staining; hematoxylin and eosin staining, SASP; senescence-associated secretary phenotype, PBS; phosphate buffered saline.

Materials—Human ADSCs and BMSCs were purchased from Lonza (Basel, Switzerland) and human umbilical vein endothelial cells (HUVECs) were a generous gift from Dr. Munehide Matsuhisa, Tokushima University, Japan. VitroGel-RGD PLUS was purchased from The Well Bioscience (New Brunswick, NJ, USA). Nanofiber bacterial cellulose (3D-NANOFIBGROW-I) gels were purchased from Nano T-Sailing (Tokushima, Japan). Click-iT EdU imaging kit was purchased from Invitrogen (Waltham, MA, USA). ELISA kits for IL-6 and MCP-1 were from R&D Systems (Minneapolis, MN, USA) and ELISA kit for IDO was from Abcam (Cambridge, UK). Cell Counting Kit-8 was purchased from Dojindo (Kumamoto, Japan). DiI was purchased from Invitrogen (Waltham, MA). C57BL/6 mice were purchased from Charles River Laboratories (Wilmington, MA, USA). LPS from Escherichia coli (serotype O111: B4) was purchased from Sigma-Aldrich (St. Louis, MO, USA). EGM-2 medium and supplements were purchased from Lonza (Basel, Switzerland). Anti-CD31 antibodies was purchased from PROTEINTECH (Rosemont, IL) and anti-CD90 antibodies was purchased from Lifespan Biosciences (Seattle, WA). All other chemicals were of analytical grade.

Cell culture—ADSCs and BMSCs were maintained in low glucose Dulbecco's modified medium supplemented with 10% fetal bovine serum on tissue culture plastic dishes. In at least Examples 13-21, the stiffness of both VitroGel RGD-PLUS and NFBC was between 150 and 750Pa according to the manufacturers' information. HUVECs were maintained in EGM-2 medium with supplements recommended by the manufacturer, but switched to Dulbecco's modified medium (low glucose) supplemented with 10% fetal bovine serum, when high glucose treatment or high mannitol treatment was initiated.

For 35 mM or 50 mM glucose treatment, glucose was added to low glucose Dulbecco's modified medium (5 mM glucose) supplemented with 10% fetal bovine serum so that the final concentration of glucose reached 35 mM or 50 mM, respectively. For 5 mM glucose+30 mM mannitol treatment, mannitol was added to low glucose Dulbecco's modified medium (5 mM glucose) supplemented with 10% fetal bovine serum so that the final concentration of mannitol reached 30 mM.

Preparation of 250 Pa VitroGel-RGD PLUS gels and 3D-NANOFIBGROW-I gels with/without ADSCs suspended in phosphate buffered saline (PBS) in them was carried out according to the manufacturers' instructions.

Quasi-3D culture—Substrate sandwiches to mimic a three-dimensional (3D) environment were composed as described previously (Winer J P, Janmey P A, McCormick M E, Funaki M (2009) Bone marrow-derived human mesenchymal stem cells become quiescent on soft substrates but remain responsive to chemical or mechanical stimuli. Tissue Eng Part A 15:147-154) with some minor modifications. Either BMSCs or ADSCs were seeded onto a 6-well plate covered with VitroGel-RGD PLUS at a cell density of 1.25-5×10\hat{ }4 cells per well. After 24 hours of culture, excess medium was removed, and a glass coverslip was placed on top of the seeded gel. To bring the cells into close proximity with a coverslip, a sterilized 35-g weight was placed on top of the sandwich for 60 seconds. The medium was reintroduced after the weight was removed, and the cells were left in the sandwiches for 24 hours. The cells were imaged or subjected to either a proliferation assay or a measurement of dehydrogenase activity.

For high glucose treatment, glucose was added to low glucose Dulbecco's modified medium (5 mM glucose) supplemented with 10% fetal bovine serum so that the final concentration of glucose reached 35 mM. For mannitol treatment, mannitol was added to low glucose Dulbecco's modified medium (5 mM glucose) supplemented with 10% fetal bovine serum so that the final concentration of mannitol reached 30 mM.

Preparation of VitroGel-RGD PLUS gels and 3D-NANOFIBGROW-I gels with/without cells suspended in phosphate buffered saline (PBS) in them was carried out according to the manufacturers' instructions.

Animal experiment—The experimental protocol was approved by the Ethics Review Committee for Animal Experimentation at Tokushima University. For injecting lipopolysaccharide (LPS), 1 mg/ml LPS solution was prepared with normal saline. 8-12 week-old male mice were intraperitoneally injected with 5 mg/kg LPS, as reported previously (Hwang J S, Kim K H, Park J, Kim S M, Cho H, et al. (2019) Glucosamine improves survival in a mouse model of sepsis and attenuates sepsis-induced lung injury and inflammation. J Biol Chem 294: 608-622). For Examples 13-21, 8-12 week-old C57BL/6 male mice were subcutaneously injected with approximately 300 μl gels or gels containing either ADSCs or BMSCs. Prior to subcutaneous injection, cells were labeled with DiI according to the manufacturers' instructions.

Imaging—Immunostaining was performed as described previously (Funaki M, Randhawa P, Janmey PA (2004) Separation of insulin signaling into distinct GLUT4 translocation and activation steps. Mol Cell Biol 24: 7567-7577).

For proliferation assay, cells were incubated in the presence of EdU overnight (or 24 hours for Examples 13-21) and EdU incorporation was visualized and compared with the images of Hoechst 33342 staining, which visualize nuclei, according to the manufacture's instruction. Hematoxylin and eosin staining was conducted at the Support Center for Advanced Medical Sciences, Tokushima University Graduate School of Biomedical Sciences. All images were obtained on a Keyence BZ-X800 microscope (Osaka, Japan). Images for each sample were obtained in at least three randomly chosen fields.

ELISA (IDO, IL-6, MCP-1)—10⁴ ADSCs either directly seeded on the bottom of a plate or embedded in gels were prepared in each well of a 96-well tissue culture plastic plate and cultured in 200 μl medium. Cells were treated as described in the Figures. For IDO ELISA, medium was removed after treatment and cell extracts were prepared and subjected to ELISA according to the manufacturer's instruction. For IL-6 and MCP-1 ELISA, 100 μl of supernatant were collected from each sample, centrifuged at 1000 g to remove cellular debris, and mixed with 100 μl of fresh medium, which was then subjected to ELISA according to the manufacturers' instructions.

Viability assay—10³ HUVECs were seeded per each well in a 96-well tissue culture plate. After being either left untreated in the medium or treated with high glucose or high mannitol followed by treatment with conditioned medium from ADSCs as described in the Figures, cell viability was measured using WST-8 assay by utilizing Cell Counting Kit-8, as described previously (Lei L T, Chen J B, Zhao Y L, Yang S P, He L (2016) Resveratrol attenuates senescence of adipose-derived mesenchymal stem cells and restores their paracrine effects on promoting insulin secretion of INS-1 cells through Pim-1. Eur Rev Med Pharmacol Sci 20:1203-1213).

Statistical analysis—P-values were calculated using an unpaired Student t-test; values of p<0.05 were considered significant.

### Example 1: 3D Culture of ADSCs in Soft Gels Made ADSCs Quiescent

To determine if ADSCs become quiescent in soft gels, 250 Pa VitroGel-RGD PLUS containing ADSCs were prepared and cultured in the presence of serum.

ADSCs were either seeded on tissue culture plates (Tissue Culture Plate) or embedded in 250 Pa VitroGel-RGD PLUS gels. The morphology of ADSCs were evaluated by a phase contrast microscopy (top panels). Proliferation assay was conducted as described in paragraph above. EdU-positive cells (middle panels) and Hoeshcst33342 staining, which shows all nuclei (lower panels), were compared in the same field. Representative images from one experiment are shown and similar results were obtained in two other independent experiments.

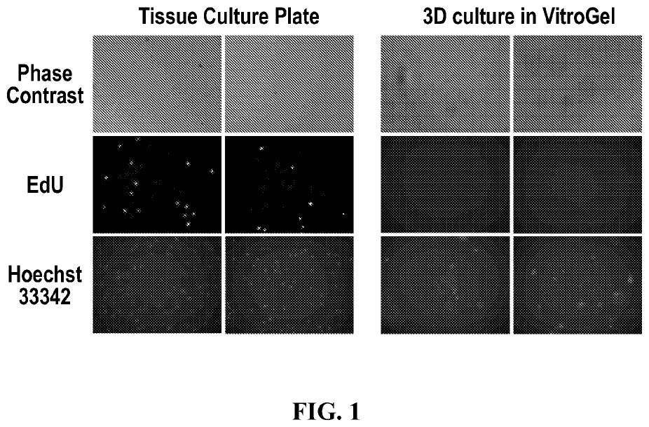

When ADSCs were seeded on a plastic tissue culture plate as a control, they exhibited a spindle shape and approximately 20% of nuclei were positive for EdU, indicating the population of cells proliferated during the overnight incubation in the presence of EdU (FIG. 1). On the other hand, cells in gels exhibited a round shape and no EdU uptake was observed, which is the same feature as quiescent bone marrow-derived mesenchymal stem cells seeded on the surface of 250 Pa polyacrylamide gels reported previously (Winer JP, Janmey PA, McCormick ME, Funaki M (2009) Bone marrow-derived human mesenchymal stem cells become quiescent on soft substrates but remain responsive to chemical or mechanical stimuli. Tissue Eng Part A 15:147-154). These results suggest that 3D culture of ADSCs in soft gels made these cells quiescent.

### Example 2: Quiescent ADSCs Exhibited Superior Anti-Inflammatory Factor Expression Over Non-Quiescent ADSCs in an In Vitro Inflammatory Model

To compare anti-inflammatory functions in vitro between quiescent ADSCs and non-quiescent ADSCs, expression levels of IDO, which has been known to play a major role in the anti-inflammatory functions of ADSCs, were investigated.

To mimic inflammatory environment in patients, cells were treated with a combination of TNFα and IFNγ. ADSCs were seeded either on plastic tissue culture plates (Tissue Culture Plate) or in 3D-NANOFIBGROW-I gels (Gel) and either left untreated or treated with 20 ng/ml TNFα+20 ng/ml IFNγ for the indicated period. For ‘Control,’ medium without cells was placed in a tissue culture plate. The concentration of IDO in cell extracts were measured by ELISA as described in paragraph above. Data are expressed as means±standard deviation of triplicates. Similar results were obtained in two other independent experiments.

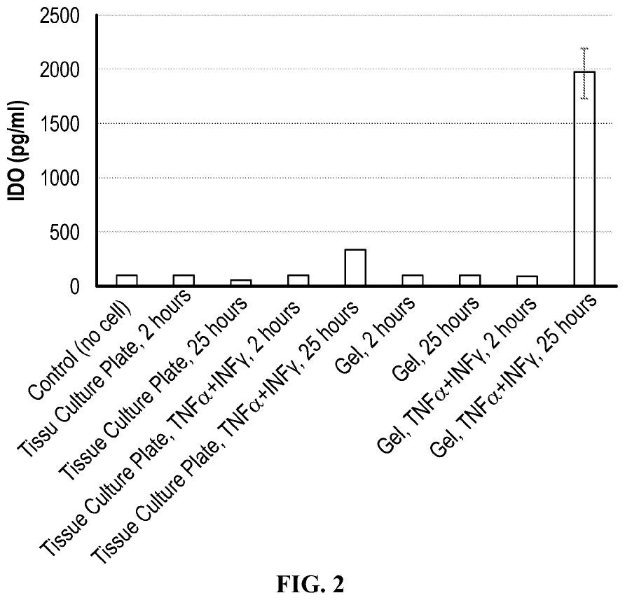

As shown in FIG. 2, treatment of non-quiescent ADSCs with TNFα plus IFNγ for 25 hours significantly upregulated IDO expression. The same treatment further drastically upregulated IDO expression in quiescent ADSCs. These results suggest that quiescent ADSCs may exhibit higher anti-inflammatory functions than non-quiescent ADSCs in an inflammatory environment in vitro.

### Example 3: Soft Environment-Induced Quiescence Restores Anti-Inflammatory Functions of ADSCs After Going Through Premature Senescence by Oxidative Stress

Oxidative stress has been known to cause premature senescence in vitro, which could be one of the underlying mechanisms of age-related diseases and conditions. Hydroxyperoxide treatment and high glucose treatment cause oxidative stress in MSCs. Thus, non-quiescent ADSCs on tissue culture plates were treated with either hydroxyperoxide (H₂O₂) or high glucose.

ADSCs were seeded on plastic tissue culture plates and either left untreated (vehicle) or treated with 200 μM H₂O₂ for two hours and then switched to a normal medium (H₂O₂), or treated with 50 mM glucose (50 mM glucose). 24 hours after initiating each treatment, cells were either left on tissue culture plates (TC) or harvested and embedded in 3D-NANOFIBGROW-I gels (TC-Gel). Cells were then either left untreated (vehicle, H₂O₂) or kept treated with 50 mM glucose (50 mM glucose). After additional 24 hours of incubation, IL-6 concentration in each medium was measured. Data are expressed as means±standard deviation of duplicates.

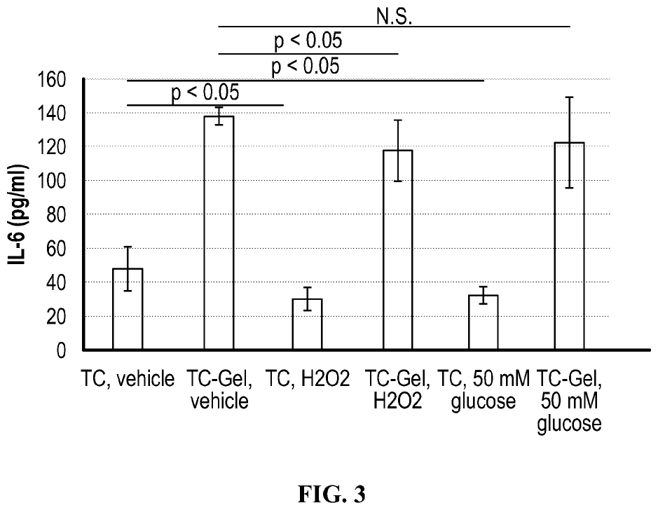

Some cells were harvested and embedded in gels to make them quiescent. Secretion level of IL-6 was compared between non-quiescent ADSCs and quiescent ADSCs converted from a non-quiescent state, since a transiently increased IL-6 secretion by MSCs plays a crucial role in suppressing inflammation and wound healing (FIG. 3).

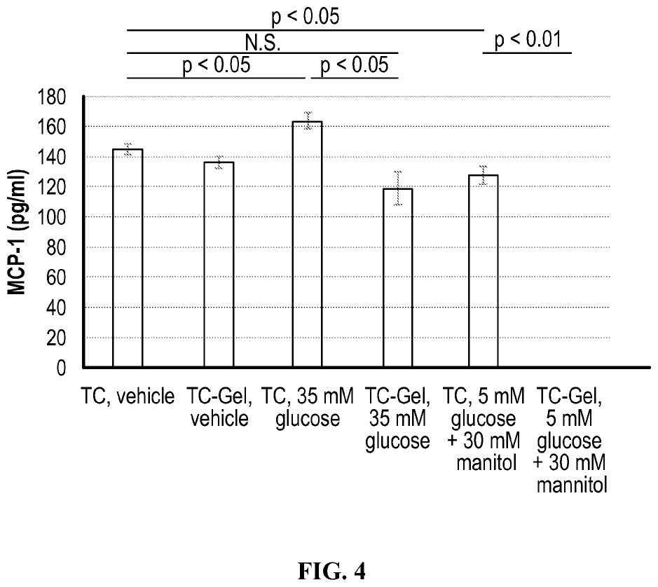

Secretion level of MCP-1 was also compared between non-quiescent and ‘quiescent now but once non-quiescent’ ADSCs, since MCP-1 was reported as a dominant chemokine in SASP (FIG. 4). ADSCs were seeded on plastic tissue culture plates and either left untreated (vehicle) or treated with 35 mM glucose (35 mM glucose) or with 5 mM glucose plus 30 mM mannitol (5 mM glucose+30 mM mannitol). 24 hours after initiating each treatment, cells were either left on tissue culture plates (TC) or harvested and embedded in 3D-NANOFIBGROW-I gels (TC-Gel). Cells were then either left untreated (vehicle) or kept treated with 35 mM glucose (35 mM glucose) or with 5 mM glucose plus 30 mM mannitol (5 mM glucose+30 mM mannitol). After additional 24 hours of incubation, MCP-1 concentration in each medium was measured. Data are expressed as means±standard deviation of duplicates. Similar results were obtained in two other independent experiments.

As shown in FIG. 3, both hydroxyperoxide treatment and high glucose treatment significantly attenuated IL-6 secretion by non-quiescent ADSCs. However, a quiescence state converted from a non-quiescent state restored IL-6 secretion mostly for hydroxyperoxide treatment and completely for high glucose treatment. As shown in FIG. 4, high glucose treatment significantly increased MCP-1secretion by non-quiescent ADSCs, but a quiescence state converted from a non-quiescent state completely abrogated high glucose-induced increase in MCP-1 secretion. Interestingly, high mannitol treatment significantly decreased MCP-1 secretion by non-quiescent ADSCs and completely eliminated MCP-1 secretion from quiescent ADSCs converted from a non-quiescent state. High glucose treatment causes not only oxidative stress but also hyperosmotic shock. Thus, cells treated with high level of mannitol were also prepared, since mannitol only causes hyperosmotic stress without oxidative stress and may enable to find out if the effect of high glucose treatment is attributable to oxidative stress or hyperosmotic stress. In this experiment ADSCs responded completely in opposite directions; high glucose treatment stimulated MCP-1 secretion, while high mannitol treatment suppressed it. Nevertheless, the results still demonstrate that oxidative stress under high glucose environment enhances MCP-1 secretion by non-quiescent ADSCs, which is inhibited by converting cells from a non-quiescent state to a quiescence state.

These results suggest that introducing and maintaining quiescence causes ADSCs to recover from oxidative stress-induced SASP, even when oxidative stress is still persisting.

### Example 4: Conditioned Medium From Quiescent ADSCs Converted From a Non-Quiescent State Led Higher Viability of HUVECs Than Conditioned Medium From Non-Quiescent ADSCs Under Oxidative Stress

To investigate the effect of secretome and extracellular vesicles secreted from ADSCs under oxidative stress on neighboring cells, conditioned medium from ADSCs were collected and administrated on HUVECs, an endothelial cell line. As an in vitro model of oxidative stress-related vascular diseases to test an ability of ADSCs for their treatment, HUVECs were also subjected to high glucose or high mannitol treatment.

ADSCs were seeded on plastic tissue culture plates and either left untreated (vehicle) or treated with 200 μM H₂O₂ for two hours and then switched to a normal medium (H₂O₂), or treated with 35 mM glucose (35 mM glucose) or with 5 mM glucose plus 30 mM mannitol (5 mM glucose+30 mM mannitol). 24 hours after initiating each treatment, cells were either left on tissue culture plates (TC) or harvested and embedded in 3D-NANOFIBGROW-I gels (TC-Gel). Cells were then either left untreated (vehicle, H₂O₂) or kept treated with 35 mM glucose (35 mM glucose) or with 5 mM glucose plus 30 mM mannitol (5 mM glucose+30 mM mannitol). After additional 24 hours of incubation, medium from each sample was collected and used for HUVECs. HUVECs were seeded on plastic tissue culture plates and either left untreated (vehicle, H₂O₂) or treated with 35 mM glucose (35 mM glucose) or with 5 mM glucose plus 30 mM mannitol (5 mM glucose+30 mM mannitol). 24 hours after initiating each treatment, medium was switched to conditioned medium from ADSCs. After three days of additional incubation, the viability of HUVECs was evaluated as described in paragraph above. Data are expressed as means±standard deviation of triplicates. Similar results were obtained in two other independent experiments.

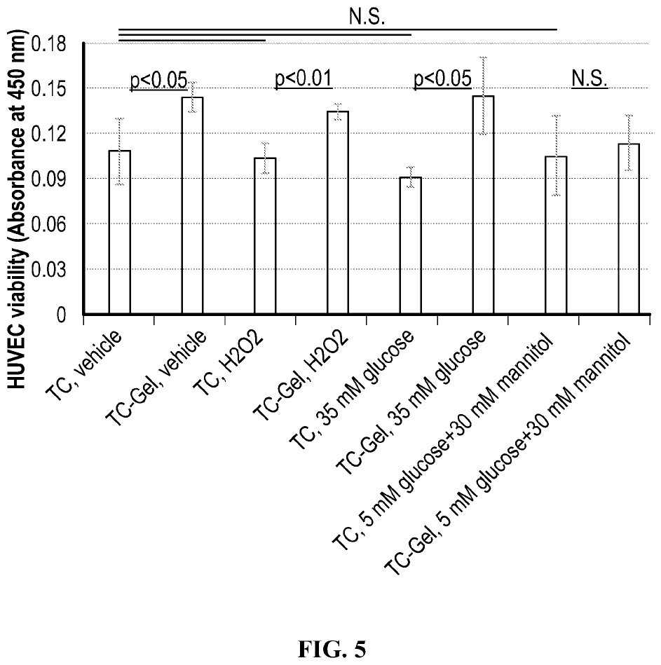

As shown in FIG. 5, HUVECs exhibited a tendency of lower viability after high glucose treatment, although the difference did not reach statistically significant. When conditioned medium from either untreated ADSCs or hydroxyperoxide-treated ADSCs was administrated on untreated HUVECs, conditioned medium from quiescent ADSCs converted from a non-quiescent state exhibited significantly higher viability of HUVECs over conditioned medium from non-quiescent ADSCs. Furthermore, when conditioned medium from high glucose-treated ADSCs was administrated on high glucose-treated HUVECs, again conditioned medium from quiescent ADSCs converted from a non-quiescent state exhibited significantly higher viability of HUVECs over conditioned medium from non-quiescent ADSCs. When conditioned medium from high mannitol-treated ADSCs, both quiescent and non-quiescent, on high mannitol-treated HUVECs was challenged, the viability of HUVECs were similar to that of untreated HUVECs with conditioned medium from untreated ADSCs. These results suggest that non-quiescent ADSCs are susceptible to oxidative stress-induced SASP. However, such SASP could be restored by converting ADSCs to a quiescent state and secretome and extracellular vesicles secreted from those ADSCs may be beneficial for endothelial cell survival even under oxidative stress, which supports the idea of applying ADSCs induced and maintained in quiescence to age-related diseases and conditions, no matter what the original condition of ADSCs is.

### Example 5: Intraperitoneal Injection of ADSCs Embedded in Gels Were Identified at Least Seven Days Later and Were Effective in Preventing LPS-Induced Acute Lung Injuries

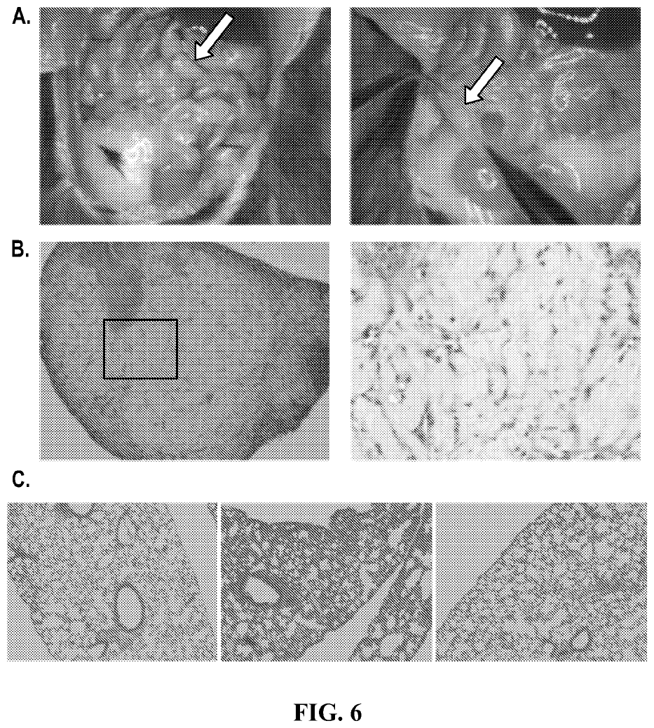

The idea of applying quiescent ADSCs to age-related diseases and conditions was tested in vivo. As shown in FIGS. 6A and B, gels intraperitoneally injected into mice were still visible and cells in those gels could be identified seven days after injection. Furthermore, when mice were intraperitoneally administrate with LPS with/without ADSC, little locomotive activity was observed in mice without ADSC injection, whereas high locomotive activity was observed in mice with ADSC injection, two days later. Seven days after intraperitoneal administration of LPS, alveolar wall became thick and infiltration of immune cells were apparent, which were completely prevented if quiescent ADSCs were intraperitoneally injected at the same time of intraperitoneal administration of LPS (FIG. 6C). These results suggest that quiescent ADSCs in biocompatible gels may locally stay at least for seven days in vivo and suppress inflammation in remote tissues and organs.

The immunomodulatory and anti-inflammatory functions of MSCs, including ADSCs, are expected to exert therapeutic effects on a wide variety of age-related diseases and conditions. However, it has been found that MSCs actually become pro-inflammatory, instead, when they are exposed to an inflammatory environment for a prolonged period. SASP of MSCs under an inflammatory environment is a serious challenge for clinical applications of MSC-based cell therapy, since affected tissues and organs are basically inflamed, which may be one of the reasons for insufficient efficacy of MSC-based cell therapies. In fact, non-quiescent ADSCs cultured on plastic tissue culture plates exhibited stress-induced SASP (FIGS. 3-5). However, introduction and maintenance of quiescence in those once non-quiescent cells successfully eliminated SASP even in the presence of conditions causing oxidative stress. Thus, whether or not ADSCs show SASP due to premature senescence, they could exert therapeutic effects for age-related diseases and conditions once they become quiescent. Accordingly, even ADSCs isolated from patients, which are supposedly inflamed and showing SASP, could serve to treat inflammatory environment, once they are maintained or induced to be in a quiescent state.

Conditioned medium from quiescent ADSCs, which contains secretome and extracellular vesicles originated from these cells, effectively rescued endothelial cells from damages caused by oxidative stress (FIG. 5). Oxidative stress-induced endothelial dysfunction is one of early events that lead to age-related diseases. Thus, favorable effects of soluble factors from quiescent ADSCs on endothelial cells under oxidative stress in vitro open up a possibility of quiescent ADSC-based cell therapy for age-related diseases and conditions in clinical settings. Furthermore, the data demonstrated that improved viability of HUVECs by conditioned medium from quiescent ADSCs were apparent, even when HUVECs are still exposed to conditions causing oxidative stress, such as high glucose concentrations (FIG. 5). Thus, quiescent ADSC-based cell therapy may bring solutions for age-related diseases and conditions, even when pathophysiological mechanisms underlying such conditions are still ongoing, which could be occasionally difficult to get rid of. For instance, diabetic angiopathies, both macro and micro, could be cured without a necessity of appropriate glycemic control. Further in vivo and clinical research is necessary to explore this possibility.

In a previous study it was demonstrated that bone marrow-derived mesenchymal stem cells became quiescent on the surface of 250 Pa polyacrylamide gels. In this study a goal was to investigate if quiescent MSCs have clinical relevance. To this end, 3D biocompatible commercially available soft gels as a substrate for ADSCs were adopted. In 3D 250 Pa VitroGel-RGD PLUS gels ADSCs exhibited the same features as quiescent bone marrow-derived MSCs on 250 Pa polyacrylamide gels, such as a round shape, lack of stress fibers and cell cycle arrest (FIG. 1). ADSCs in 3D-NANOFIBGROW-I gels also exhibited a round shape (FIG. 6B) and apparently eliminated SASP due to premature senescence, which cells encountered before embedded in 3D-NANOFIBGROW-I gels (FIGS. 3-5). Thus, it would be safe to say that quiescent MSCs on 2D soft gels were successfully reproduced in 3D biocompatible gels, which makes it possible to investigate if quiescent MSCs have clinical relevance.

High glucose treatment causes both oxidative stress and hyperosmotic stress and hyperosmotic stress has been reported to induce some cellular responses in both MSCs and HUVECs. In order to find out if cellular responses observed by high glucose treatment is attributable to high glucose-induced oxidative stress, cells treated with high glucose were compared with cells treated with high mannitol, which is known to cause only hyperosmotic shock (FIGS. 3 and 4). Although high mannitol treatment did not affect HUVECs' viability during the time frame of the experiments, it indeed decreased MCP-1 secretion by both non-quiescent and quiescent ADSCs converted from a non-quiescent state. The mechanism and significance of hyperosmorality to inhibit MCP-1 secretion by ADSCs are unclear at this time. However, such inhibition can lead to an underestimate of oxidative stress-induced MCP-1 secretion by ADSCs, which is a major feature of SASP in premature senescence of ADSCs. As hyperosmolarity-induced reduction in MCP-1 secretion was more drastic in non-quiescent ADSCs, the reduction in MCP-1 secretion by introducing and maintaining quiescence into non-quiescent ADSCs, which supposedly had SASP under a high glucose environment, should be more remarkable than that is observed in FIG. 4.

A prevailing view of SASP is that senescent cells affect the functions of their neighboring cells. If quiescent ADSCs were to be used for a disease treatment, ADSCs may be administrated with gels as their scaffold in order to make ADSCs quiescent, instead of administrating ADSCs without a scaffold intravenously or locally (e.g., intramuscularly or intratracheally), which has been vigorously tested for clinical applications until today. There are apparent advantages of using gels as a scaffold; rapid disappearance of cells is one of major challenges for present MSC-based cell therapies, in which cells are administrated without a scaffold. To overcome this issue, a large amount of MSCs are used for each treatment, which necessitates a vast ex vivo expansion of MSCs. Furthermore, most intravenously injected MSCs are trapped in lung capillaries, instead of being targeted to inflamed tissues/organs, which raises a concern for pulmonary embolism due to administration of a large amount of MSCs. ADSCs embedded in gels can be injected as was done in FIG. 6, which shows that ADSCs stay in gels for a prolonged period. Thus, loss of cells may not be a concern and administrating a large amount of ADSCs is not necessary, which should make ADSC-based cell therapy safer and easier to access for patients. One potential drawback of administrating ADSCs in gels is that the inflamed tissues and organs in patients may not be necessarily in a close proximity of administrated ADSCs. Inflammation could be even systemic. Thus, we investigated whether or not locally administrated ADSCs in gels to introduce and maintain quiescence in them are able to suppress inflammation remotely. To this end, an acute lung injury model was adopted, which is also drawing much attention recently as a model for lung injuries in COVID-19, by intraperitoneally injecting low dose of LPS into mice (FIG. 6C). Surprisingly, intraperitoneally administrated ADSCs in gels successfully maintained locomotive activities of mice even after LPS administration and prevented LPS-induced acute lung injuries. Thus, embedding ADSCs in gels and placing them in vivo may not only make ADSCs quiescent and cancel any existing premature senescence, but also control inflammation in remote tissues and organs.

In conclusion, SASP of ADSCs due to their premature senescence caused by oxidative stress can be eliminated, when quiescence is introduced and maintained in ADSCs. Quiescent ADSCs converted from a non-quiescent state are able to exert ant-inflammatory functions not only in their neighboring areas but also in remote areas. Thus, converting ADSCs from a non-quiescent state to a quiescent state may rejuvenate ADSCs themselves, their neighboring cells, and cells in remote tissues and organs.

### Example 6: MSCs on Soft Substrates Stayed Quiescent in a Pro-Inflammatory Environment

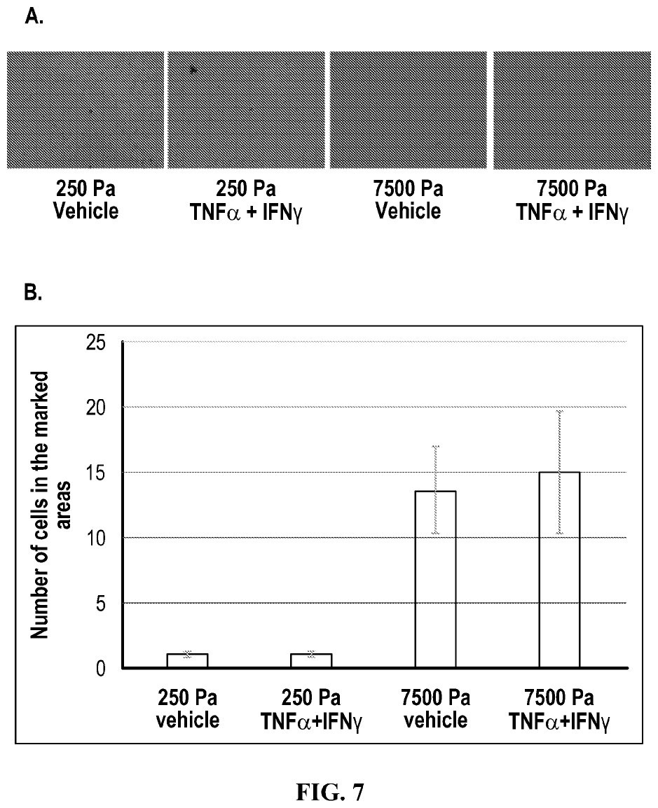

In this study the clinical relevance of quiescent MSCs was investigated. As MSCs are supposed to suppress inflamed tissues and organs in patients in MSC-based cell therapies, whether MSCs stay quiescent on 250 Pa gels in a pro-inflammatory environment in vitro is investigated. To this end, the same number of MSCs were seeded on either 250 Pa or 7500 Pa polyacrylamide gels and they are incubated in the absence or presence of TNFα and IFNγ, which has been used as an in vitro pro-inflammatory environment in patients. As shown in FIG. 7, treatment of MSCs with a combination of TNFα and IFNγ failed to affect the number of MSCs both on 250 Pa gels and 7500 Pa gels. These results implicate that MSCs stay quiescent on 250 Pa gels even in a pro-inflammatory environment.

### Example 7: Quiescent MSCs Were Able to Upregulate Factors Involved in the Modulatory Functions Even in a Pro-Inflammatory Environment

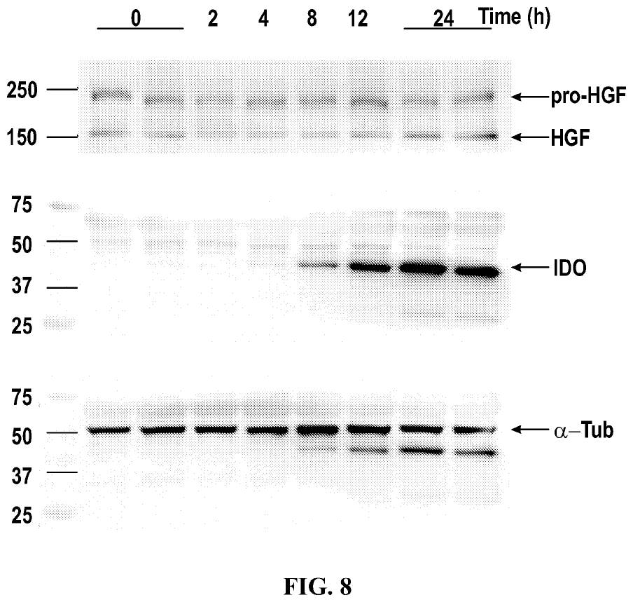

It was then investigated whether or not quiescent MSCs are capable of producing factors responsible for their immunomodulatory functions in a pro-inflammatory environment in vitro. To this end, MSCs seeded on the surface of 250 Pa polyacrylamide gels were treated with a combination of TNFα and IFNγ for the indicated time. As shown in FIG. 8, expression levels of HGF and IDO, which have been known to play a major role in the anti-inflammatory functions of ADSCs, were remarkably upregulated by TNFα plus IFNγ stimulation in a time dependent manner. These results suggest that MSCs are capable of producing factors involved in their immunomodulatory functions, even when they are quiescent.

### Example 8: Quiescent MSCs Exhibited Immunomodulatory Functions In Vivo in a Sjöegren's Syndrome Model

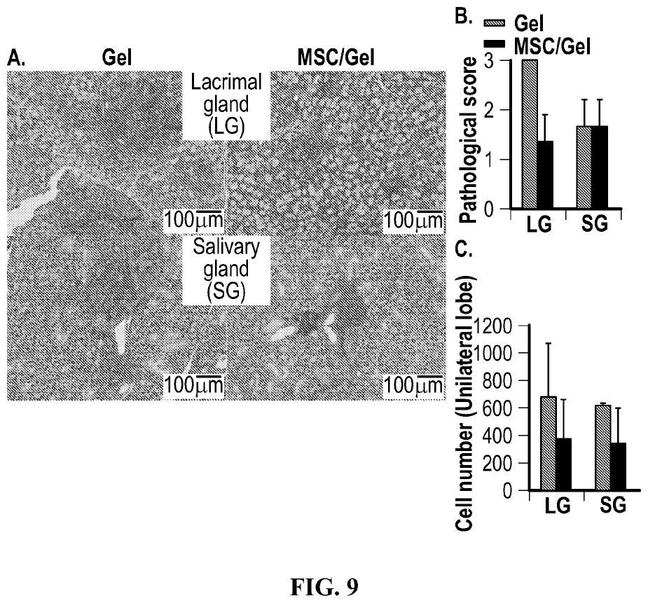

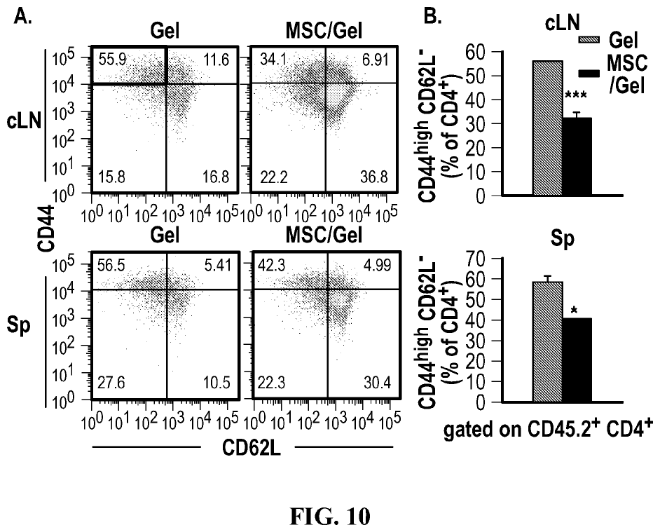

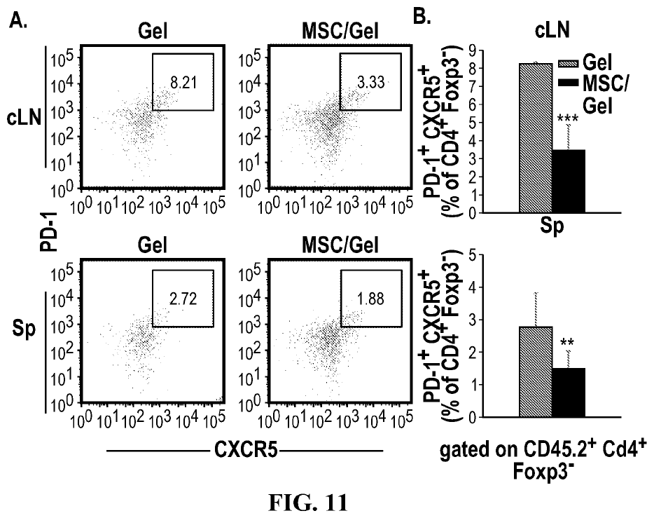

To investigate the immunomodulatory functions of quiescent MSCs in vivo, Sjöegren's syndrome model mice were used. Sjöegren's syndrome is one type of autoimmune diseases, which shows chronic inflammation in multiple exocrine glands, such as lacrimal glands and salivary glands. As shown in FIG. 9, intraperitoneal administration of quiescent MSCs embedded in VitroGel RGD-PLUS led to a significantly lower Pathological score in lacrimal glands. The number of infiltrated lymphocytes both in lacrimal glands and salivary glands were significantly lower in mice administrated with quiescent MSCs in gels. Furthermore, administration of quiescent MSCs in gels led to a significant decrease in the population of CD62L(low)CD44(high) CD4(+) T cells (FIG. 10) and PD-1(high)CXCR5(high) CD4(+) T cells (FIG. 11), which correspond to effector memory T cells and follicular helper T cells, respectively, both in cervical lymph nodes and in the spleens. These results suggest that quiescent MSCs are capable of exerting their immunomodulatory functions in vivo.

### Angiogenesis

Blood supply through newly formed blood vessels into a transplanted gel is expected to promote survival of implanted ADSCs, which should in turn lead to higher efficacy of ADSC-based cell therapy. As ADSCs have been known to promote angiogenesis, ADSCs embedded in a gel causes angiogenesis in vivo. 300 μl 3D-NANOFIBGROW-I gel with either vehicle (PBS) or 3×10⁶ ADSCs is prepared and subcutaneously administrated into a C57BL/6 male mouse. Each group consists of three mice. Three weeks later, mice are sacrificed and gels are isolated from the injection site, fixed and stained with either hematoxylin and eosin or anti-CD31 antibodies to visualize newly-formed vasculatures in the gels. Newly-formed vasculatures are detected in the gels containing ADSCs, but not in the gels prepared without ADSCs.

### Example 9: Protective Roles of Quiescent ADSCs Subcutaneously Injected With Gels Against LPS-Induced Acute Lung Injuries

Data in FIG. 6C demonstrates a protective role of quiescent ADSCs embedded in 3D-NANOFIBGROW-I against LPS-induced acute lung juries. In that experiment, ADSCs and gels were intraperitoneally administrated into mice. In a clinical setting, subcutaneous injection is easier and safer. Thus, it is expected that subcutaneously administrated quiescent ADSCs in gels also show protective effects against LPS-induced lung injuries. 300 μl 3D-NANOFIBGROW-I gels containing either vehicle (PBS) or 3×10⁶ ADSCs are prepared. Either PBS-containing gel or ADSC-containing gel is subcutaneously injected into mice. Vehicle (normal saline) or 1 mg/ml LPS dissolved in normal saline is also prepared. Either vehicle or LPS (5 mg/kg) is subcutaneously injected into C57BL/6 male mice at the same time of 3D-NANOFIBGROW-I gel injection. 21 days after injection, mice are sacrificed and lungs will be isolated for hematoxylin and eosin staining. Each group will consist of three mice. LPS causes alveolar wall thickening and infiltration of immune cells, which may be eliminated by subcutaneously administrating ADSCs embedded in 3D-NANOFIBGROW-I gels.

### Example 10: Elimination of Stress-Induced Premature Senescence and SASP in Neighboring Cells by Quiescent ADSCs in a Chronic Disease Model

One of the complications of diabetes is poor wound healing and recent evidence suggests that hyperglycemia-induced SASP in endothelial cells and macrophages is playing a role. Thus, administering quiescent ADSCs eliminates high glucose-induced premature senescence, which leads to an accelerated wound healing even under diabetic conditions. Diabetes is caused by intraperitoneally injecting streptozotocin into C57BL/6 male mice as reported previously (Pak C S, Heo C Y, Shin J, Moon S Y, Cho S W, et al. (2021) Effects of a Catechol-Functionalized Hyaluronic Acid Patch Combined with Human Adipose-Derived Stem Cells in Diabetic Wound Healing. Int J Mol Sci 22). Four weeks after streptozotocin injection, excision biopsy wounds are made on the shaved dorsal regions of the midline extending through the panniculus carnosus using a 6 mm punch. 300 μl 3D-NANOFIBGROW-I gels containing either vehicle (PBS) or 3×10⁶ ADSCs are prepared. Either PBS-containing gel or ADSC-containing gel is subcutaneously injected into an area at least 1 cm apart from the wounds of diabetic mice. Wound areas are monitored for the next 21 days. Under diabetic conditions, mice administrated with PBS-containing gels fail to show wound healing, whereas mice administrated with ADSC-containing gels show healing.

### Example 11: Rejuvenation of ADSCs Isolated From Inflamed Adipose Tissues by Introducing and Maintaining Quiescence in Them

Application of quiescent ADSCs embedded in gels to treat age-related diseases is expected to show higher efficacy than conventional ADSC-based cell therapies, in which cells are administrated intravenously or locally without a scaffold, since cells are unlikely to disappear in a short period as have been observed in conventional ADSC-based cell therapies. This advantage should enable quiescent ADSC-based cell therapies with much fewer number of cells. For instance, auto-transplant of ADSCs may become sufficient to treat age-related diseases, which should make a treatment much simpler and at a lower cost. As chronic inflammation is ongoing in patients, ADSCs may be in a premature senescent state in patients' inflamed adipose tissues and showing SASP. Thus, it is expected that that pro-inflammatory ADSCs can be rejuvenated and SASP can be eliminated, once ADSCs are isolated from patients' adipose tissues and introduced and maintained in a quiescent state in gels. ADSCs are isolated from ob/ob obese mice. Isolated ADSCs are either seeded on plastic tissue culture plates or in 3D-NANOFIBGROW-I gels. After three days of culture, conditioned medium is collected and used as a culture medium for HUVECs. The viability of HUVECs is compared between conditioned medium from ADSCs cultured on plastic tissue culture plates and conditioned medium form ADSCs cultured in gels. HUVECs cultured with conditioned medium form ADSCs cultured in gels show higher viability than HUVECs cultured with conditioned medium form ADSCs cultured on plastic tissue culture plates.

### Example 12: Range of Stiffness

The range of stiffness of gels, which show an ability to eliminate stress-induced premature senescence from ADSCs. VitroGel RGD-PLUS ranging from 150 to 750 Pa is prepared. Quiescence of ADSCs in them are determined by EdU staining and morphological observation. ADSCs embedded in 150 to 750 Pa VitroGel RGD-PLUS show no EdU uptake, a round shape and lack stress fibers.

### Example 13: Arrested Cell Cycle in BMSCs Cultured on Gels Resumed Upon Contact of Cells With Glass

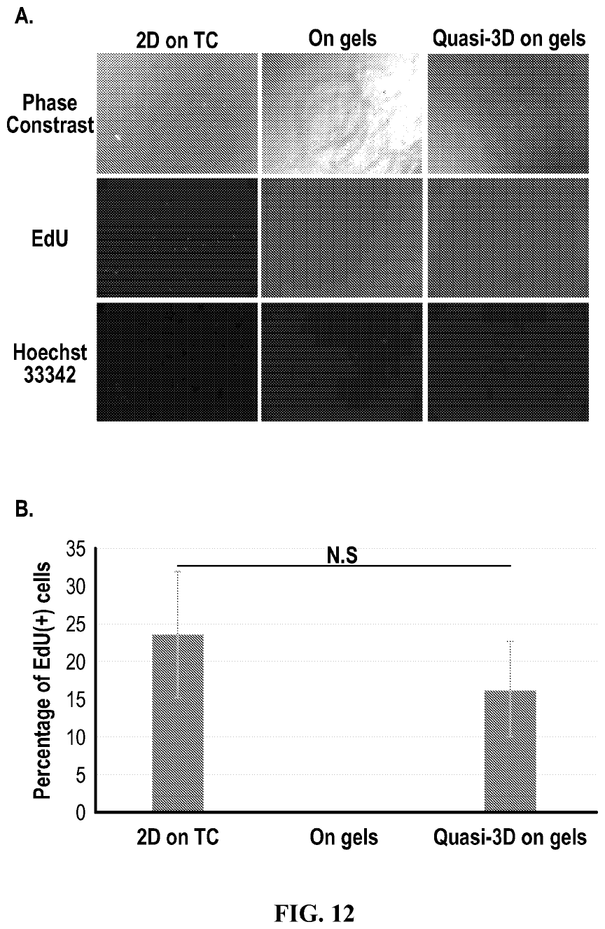

To determine that cell cycle arrest observed in BMSCs cultured on gels is reversible, BMSCs were seeded onto a plastic tissue culture plates (2D on TC) or on the surface of VitroGel RGD-PLUS. After 24 hours of incubation, cells on VitroGel RGD-PLUS were either left untreated (On gels) or subjected to a quasi-3D culture (Quasi-3D on Gels). After additional 24 hours of incubation, EdU was added to the medium and incorporation of EdU into the cells during the next 24 hours of culture was evaluated according to the manufacturer's instructions. Images were taken (FIG. 12A) and the percentage of cells positive for EdU incorporation was quantified (FIG. 12B). Data are expressed as means±standard deviation of triplicates.

As shown in FIG. 12A, BMSCs seeded on a tissue culture plastic plate (2D on TC) exhibited a spindle shape and approximately 24% of cells incorporated EdU, which indicates the population of cells proliferated during the 24 hours of incubation in the presence of EdU (FIG. 12B). BMSCs cultured on the surface of gels (On gels) exhibited a round shape and none of them incorporated EdU. Cells sandwiched between a gel and a coverslip (Quasi-3D on gels) changed their morphology and exhibited a spindle shape and approximately 16% of cells incorporated EdU, which was not statistically different from the percentage of EdU-positive cells seeded on a tissue culture plastic plate (2D on TC). These results suggest that BMSCs become quiescent on soft biocompatible gels, as were seen in BMSCs on 250 Pa polyacrylamide gels reported previously (Winer J P, Janmey P A, McCormick M E, Funaki M (2009) Bone marrow-derived human mesenchymal stem cells become quiescent on soft substrates but remain responsive to chemical or mechanical stimuli. Tissue Eng Part A 15:147-154).

### Example 14: ADSCs Cultured in 3D-NANOFIBGROW-I Gels Became Quiescent

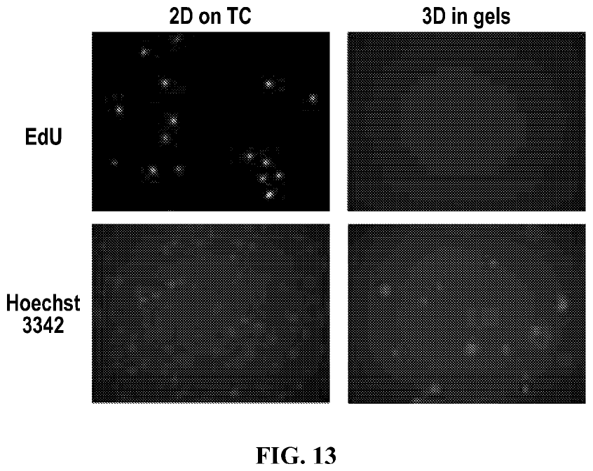

Introduction and maintenance of quiescence in MSCs were tested in 3D-NANOFIBGROW-I gels, which was chemically different from, but has similar stiffness as VitroGel RGD-PLUS gels. To this end, as shown in FIG. 13, ADSCs were either seeded on the surface of a plastic tissue culture plate (2D on TC) or embedded in 3D-NANOFIBGROW-I gels (3D in gels). ADSCs cultured in 3D in 3D-NANOFIBGROW-I gels (3D in gels) adopted a round shape and none of them incorporated EdU. These results suggest that 3D culture of MSCs in 3D-NANOFIBGROW-I gels, as well as 3D culture of MSCs in VitroGel RGD-PLUS gels, is capable of introducing and maintaining quiescence in MSCs.

### Example 15: Quiescent ADSCs Exhibited Attenuated Dehydrogenase Activity, Which was Restored Upon Contact of Cells With Glass to Make Them Non-Quiescent

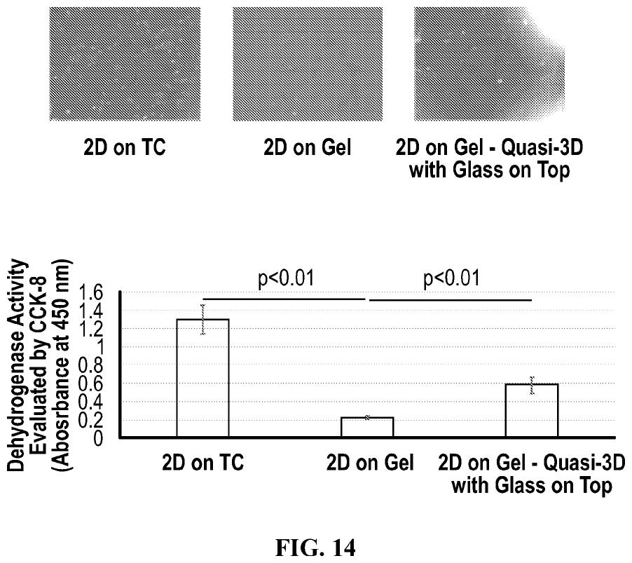

As dehydrogenases are one type of house-keeping genes, their activity is frequently considered to reflect the viability of cells, of which reduction is an irreversible process (see Li L C, Wang Z W, Hu X P, Wu Z Y, Hu Z P, et al. (2017) MDG-1 inhibits H₂O₂-induced apoptosis and inflammation in human umbilical vein endothelial cells. Mol Med Rep 16: 3673-3679; Yakisich J S, Kulkarni Y, Azad N, Iyer A K V (2017) Selective and Irreversible Induction of Necroptotic Cell Death in Lung Tumorspheres by Short-Term Exposure to Verapamil in Combination with Sorafenib. Stem Cells Int 2017: 5987015). Thus, ADSCs were seeded onto a plastic tissue culture plate (2D on TC) or on the surface of VitroGel RGD-PLUS (FIG. 14). After 24 hours of incubation, cells on VitroGel RGD-PLUS were either left untreated (2D on Gel) or subjected to a quasi-3D culture (2D on Gel-Quasi-3D with Glass on Top). After additional 24 hours of incubation, dehydrogenase activity was measured using a Cell Counting Kit-8 (CCK-8) according to the manufacturer's instructions. Data are expressed as means±standard deviation of triplicates.

As shown in FIG. 14, cells cultured on the surface of gels exhibited a significantly lower dehydrogenase activity. However, when these cells were contacted with glass, attenuated dehydrogenase activity was significantly improved, demonstrating that a reduced dehydrogenase activity in quiescent ADSCs was not an irreversible process. These results suggests that quiescence dose not impair the viability of ADSCs. The attenuated dehydrogenase activity in quiescent ADSCs may reflect an altered metabolism in these cells, which requires a further investigation.

### Example 16: Dehydrogenase Activity was Unaffected in Quiescent ADSCs in High Glucose Environment

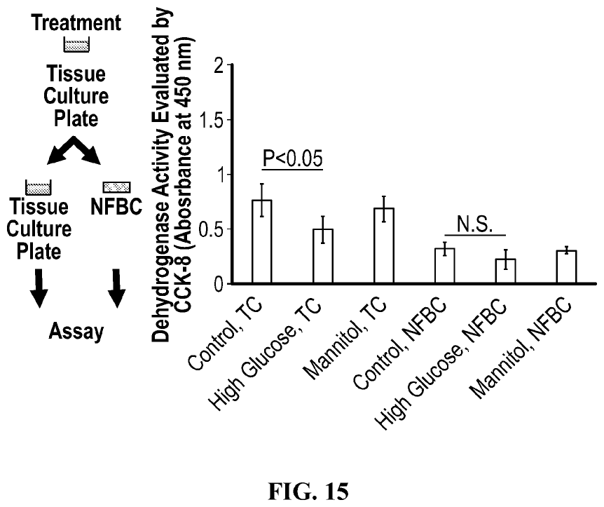

High glucose environment has been known to cause premature senescence in vitro (Zhang D, Lu H, Chen Z, Wang Y, Lin J, et al. (2017) High glucose induces the aging of mesenchymal stem cells via Akt/mTOR signaling. Mol Med Rep 16:1685-1690), which could be one of the underlying mechanisms of age-related diseases and conditions (Borodkina A, Shatrova A, Abushik P, Nikolsky N, Burova E (2014) Interaction between ROS dependent DNA damage, mitochondria and p38 MAPK underlies senescence of human adult stem cells. Aging (Albany NY) 6: 481-495). To induce oxidative stress in MSCs, non-quiescent ADSCs were treated with high glucose (FIG. 15). To this end ADSCs were seeded on plastic tissue culture plates and either left untreated (Control) or treated with either 35 mM glucose (High Glucose) or 30 mM mannitol plus 5 mM glucose (Mannitol). 24 hours after initiating each treatment, cells were either left on tissue culture plates (TC) or harvested and embedded in 3D-NANOFIBGROW-I gels (NFBC). Cells were then either left untreated (Control) or kept treated with either 35 mM glucose (High Glucose) or 30 mM mannitol plus 5 mM glucose (Mannitol). After additional 24 hours of incubation, dehydrogenase activity was measured using a Cell Counting Kit-8 (CCK-8) according to the manufacturer's instructions. Data are expressed as means±standard deviation of duplicates.

As shown in FIG. 15, high glucose treatment significantly reduced dehydrogenase activity in ADSCs on plastic tissue culture plates. As a high glucose concentration will increase the osmolality of the environment surrounding the ADSCs, which may also exert some biological effects on cells, a group of ADSCs were treated with mannitol, which will increase the osmolality but does not cause an oxidative stress observed in high glucose environment. Treating ADSCs with mannitol failed to affect dehydrogenase activity in ADSCs on plastic tissue culture plates. Thus, a lower dehydrogenase activity in ADSCs on plastic tissue culture plates under high glucose condition should reflect stress caused by high glucose concentration, but not by an elevated osmolality. When cells treated with high glucose were transferred from a plastic tissue culture plate to 3D-NANOFIBGROW-I gels, cells showed similar dehydrogenase activity as control cells even in the continued presence of high glucose. These results suggest that quiescent ADSCs become resistant to stress caused by high glucose treatment.

### Example 17: Transplanted Quiescent MSCs in Gels Remained in the Transplants and Promoted Host Cell Infiltration and Fiber Formation

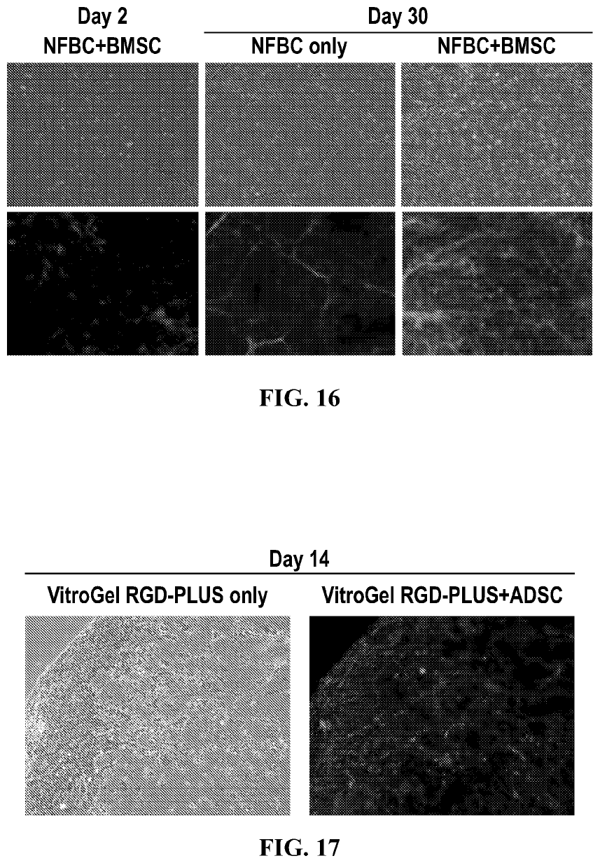

Rapid disappearance of transplanted MSCs from a host is one of the major challenges in a clinical application of MSC-based cell therapies (Braid L R, Wood C A, Wiese D M, Ford B N (2018) Intramuscular administration potentiates extended dwell time of mesenchymal stromal cells compared to other routes. Cytotherapy 20:232-244). Thus, the fate of quiescent BMSCs in gels after being transplanted into a mouse was investigated. Either 3D-NANOFIBGROW-I gels (NFBC only) or 3D-NANOFIBGROW-I gels containing approximately 2.2×10⁶ DiI-labeled BMSCs (NFBC+BMSC) were subcutaneously injected into mice. After 2 days (Day 2) or 30 days (Day 30) of transplantation, mice were sacrificed and transplanted gels with/without DiI-labeled BMSCs were isolated, which was followed by H&E staining (FIG. 16).

DiI-labeled cells were identified by a fluorescent microcopy in both gels isolated after 2 days of transplantation (Day 2, NFBC+BMSC) and gels isolated after 30 days of transplantation (Day 30, NFBC+BMSC) at a similar cell density. On the other hand, gels transplanted without BMSCs (Day 30, NFBC only) did not have cells with red fluorescence. When fluorescence images were compared with their corresponding phase contrast images, the DiI-positive cells exhibited a round shape, which is one of the features of quiescent MSCs (FIGS. 12-14). These results suggest that BMSCs transplanted in 3D-NANOFIBGROW-I gels stay in the gel for a prolonged period.

As shown in FIG. 16, both gels transplanted with BMSCs (NFBC+BMSC) and gels transplanted without BMSCs (NFBC only) had fluorescently-negative cells and fluorescently-positive fibrous structures, of which amount showed an increase in a time dependent manner, when images of Day 2 and Day 30 in gels with BMSCs were compared. The red fluorescently-positive fibrous structure was also green fluorescently-positive (data not shown), which suggest that the fluorescence signal of the structure is auto-fluorescence, instead of fluorescence associated with DiI or DiI-labeled BMSCs. The amount of both fluorescently-negative cells, which are presumably mouse cells recruited to and infiltrated into the gel, and fluorescently-positive fibrous structures in gels with BMSCs were apparently higher than those observed in gels without BMSCs. These results suggest that quiescent BMSC staying in 3D-NANOFIBGROW-I gels may promote mouse cell infiltration and fiber formation in the gels.

To confirm that prolonged detection of quiescent MSCs in gels in vivo is independent of MSC cell type or type of gels, ADSC was tested, instead of BMSC, and VitroGel RGD-PLUS gels were tested, instead of 3D-NANOFIBGROW-I gels. Either VitroGel RGD-PLUS (VitroGel RGD-PLUS only) or VitroGel RGD-PLUS containing approximately 6×10⁵ DiI-labeled ADSCs (VitroGel RGD-PLUS+ADSC) was subcutaneously injected into mice (FIG. 17). 14 days later mice were sacrificed and transplanted gels with/without DiI-labeled ADSCs were isolated, which was followed by H&E staining (FIG. 17).

As shown in FIG. 17, similar to transplants made of BMSC and 3D-NANOFIBGROW-I gels, transplants made of ADSCs and VitroGel RGD-PLUS gels contained DiI-positive round cells. Such transplants contained DiI-negative cells and autofluorescent-positive fibers, which was also observed in transplants made of BMSCs and 3D-NANOFIBGROW-I gels. These results, together with the results shown in [00144]-[00145], suggest that, when MSCs in gels are transplanted, they stay in the transplants for a prolonged period and promote host cell infiltration and fiber formation.

### Example 18: Transplanted Quiescent MSCs in Gels Promoted Angiogenesis

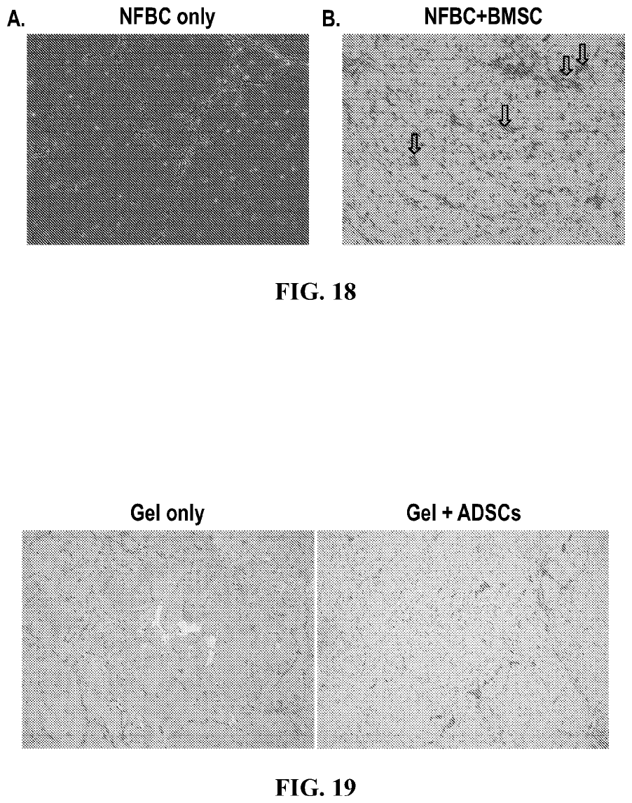

MSCs have been known to promote angiogenesis, which would contribute to exert their therapeutic effects (Watt S M, Gullo F Fau—van der Garde M, van der Garde M Fau-Markeson D, Markeson D Fau-Camicia R, Camicia R Fau—Khoo C P, et al. The angiogenic properties of mesenchymal stem/stromal cells and their therapeutic potential). Thus, whether or not MSC transplantation will enhance angiogenesis in gels, which should help transplanted cells survive and exert their functions was investigated. In FIG. 18, either 3D-NANOFIBGROW-I gels (A) or 3D-NANOFIBGROW-I gels containing approximately 2.2×10⁶ DiI-labeled BMSCs (B) were subcutaneously injected into mice. After 30 days of transplantation, mice were sacrificed and transplanted gels with/without DiI-labeled BMSCs were isolated, which was followed by H&E staining.

Vascular formation was apparent in gels containing quiescent BMSCs (allow heads in FIG. 18B) but not in gels without BMSCs (FIG. 18A). It is noteworthy that a green arrowhead in FIG. 18B is pointing at a blood vessel that contains red blood cells. These results suggest that MSCs in gels promoted angiogenesis in the transplants, which should support their own survival by increasing gas exchange and nutrient supply, as well as support exerting therapeutic effects for the hosts.

### Example 19: Transplanted Quiescent MSCs in Gels Remain as MSCs in Gels

By fluorescently-labeling MSCs before transplantation, those cells remaining in the gels for a prolonged period were identified. Those cells in gels remained as MSCs. Besides exhibiting a round shape (FIG. 16), which is one of the features of quiescent MSCs, whether or not transplanted BMSCs express CD90, one of the markers for MSCs was investigated. Either 3D-NANOFIBGROW-I gels (C) or 3D-NANOFIBGROW-I gels containing DiI-labeled BMSCs are subcutaneously injected into mice. After 30 days of transplantation, mice are sacrificed and transplanted gels with/without DiI-labeled BMSCs are isolated, which are followed by staining with either anti-CD90 antibodies or H&E (C and D).

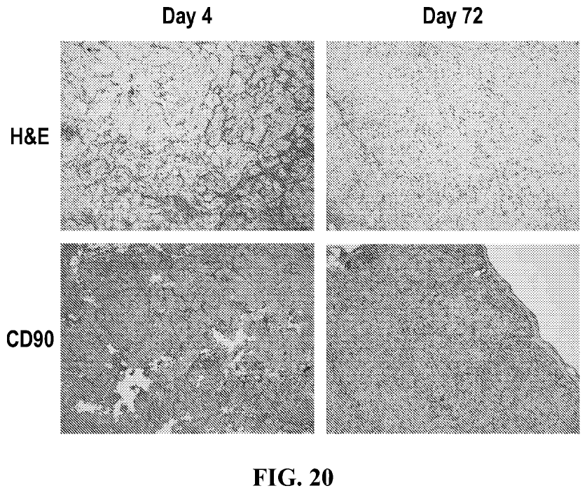

A certain population of round cells were stained with anti-CD90 antibodies. Although DiI signal was lost during the process of immunohistochemistry, the percentage of CD90-positive round cells was similar to that of DiI-positive round cells, which can be identified by a fluorescence microscopy of the same sample stained with H&E. As shown in FIG. 20, these results suggested that quiescent BMSCs in gels remain as BMSCs in the transplants.

### Example 20: Quiescent ADSCs Exhibited Superior Exosomal microRNA Secretion Over Non-Quiescent ADSCs in an In Vitro Inflammatory Model

Quiescent MSCs increase secretion of exosomal microRNA.

To compare the amount of exosomal microRNA in vitro between quiescent ADSCs and non-quiescent ADSCs, expression levels of a series of exosomal microRNA, which has been known to play a major role in the immunomodulatory or anti-inflammatory functions of ADSCs, are investigated.

To mimic inflammatory environment in patients, cells are treated with a combination of TNFα and IFNγ. ADSCs are seeded either on plastic tissue culture plates or in 3D-NANOFIBGROW-I gels. Cells are either left untreated or treated with 20 ng/ml TNFα+20 ng/ml IFNγ for 24 hours using medium supplemented with exosome-free serum to prevent a contamination of exosomes from other sources.

Then exosome is collected from the medium using a Total Exosome Isolation Reagent from Invitrogen (Waltham, MA). Total RNA is extracted from the exosome using Trizol from Invitrogen, which is subjected to a microRNA analysis. After stimulating cells with TNFα and IFNγ, exosomes derived from quiescent ADSCs contain higher amount of microRNA, which has been known to play a role in immunomodulation or anti-inflammation, over exosomes derived from non-quiescent ADSCs.

### Example 21: Quiescence Rejuvenates ADSCs From High Glucose-Induced SASP

High glucose treatment caused SASP in ADSCs, which was restored by introducing and maintaining quiescence in those cells. For instance, once high glucose-treated ADSCs were embedded in 3D-NANOFIBGROW-I gels and become quiescent, they no longer responded to high glucose treatment in their dehydrogenase activity (FIG. 15). Thus, even ADSCs in SASP condition due to a high glucose treatment can fully exert immunomodulatory and anti-inflammatory functions in vivo, once they are introduced and maintained in quiescence.

To induce SASP in ADSCs in vitro, ADSCs are cultured in a medium that contains either 5 mM glucose (normal glucose) or 35 mM glucose (high glucose).

To investigate anti-inflammatory functions of ADSCs, the process of wound healing in streptozotocin-induced diabetes mouse is compared between ADSCs cultured under normal glucose and ADSCs cultured under high glucose. Diabetes is caused by intraperitoneally injecting streptozotocin into C57BL/6 male mice as reported previously (Pak C S, Heo CA-O, Shin J, Moon S Y, Cho S W, et al. Effects of a Catechol-Functionalized Hyaluronic Acid Patch Combined with Human Adipose-Derived Stem Cells in Diabetic Wound Healing. LID—10.3390/ijms22052632 [doi] LID—2632).

### Example 22: Lack of Thrombosis in Mice Administrated With Quiescent MSCs in Gels

Transplants containing quiescent MSCs were analyzed for thrombus formation after keeping them in the subcutaneous tissues for a prolonged period. As shown in the right panel of FIG. 18, in which 3D-NANOFIBGROW-I gels containing 2.2×10⁶ DiI-labeled BMSCs were excised after 30 days of transplantation, and in the right panel of FIG. 19, in which 3D-NANOFIBGROW-I gels with 6.5×10⁵ ADSCs were excised after 72 days of transplantation, no thrombus was observed in capillaries in those gels. These results suggest that administrating quiescent MSCs in gels does not cause thrombosis, which is one of the complications accompanied by the present MSC-based cell therapies.

### Example 23: Enhanced Angiogenesis by Quiescent ADSCs Transplanted in Gels

Enhanced angiogenesis by quiescent ADSCs in gels were investigated by detecting endothelial cells, which line the interior surface of blood vessels.

500 μl 3D-NANOFIBGROW-I gel with either vehicle (PBS) or 6.5×10⁵ ADSCs was prepared and subcutaneously administrated into a C57BL/6 male mouse. Each group consisted of three mice. 72 days later, mice were sacrificed and gels were isolated from the injection site, fixed and stained with anti-CD31 antibodies, which detect expression of CD31, an established endothelial cell marker, to visualize newly-formed vasculatures in the gels.

As shown in FIG. 19, gels without ADSCs (Gel only) failed to show CD31-positive cells in them, whereas gels containing ADSCs (Gel+ADSCs) showed CD31-positive cells forming tubular structures. These data, together with data shown in FIG. 18, demonstrate that quiescent ADSCs transplanted in gels enhance angiogenesis in vivo.

### Example 24: Transplanted Quiescent MSCs in Gels Remain as MSCs In Vivo

By fluorescently-labeling MSCs before transplantation, those cells were detected by their fluorescence signal, and FIG. 16 showed that transplanted MSCs in gels stayed in gels for a prolonged period. The remaining cells in the gels shown in FIG. 16 exhibited a round shape, which is one of the features of quiescent MSCs. To further establish that cells in gels are indeed maintained as MSCs, Gels transplanted with ADSCs were kept in mouse tissues for a prolonged period, which were then excised and stained for CD90, a marker for MSCs.

500 μl 3D-NANOFIBGROW-I gel with 6.5×10⁵ ADSCs was prepared and subcutaneously administrated into a C57BL/6 male mouse. Either 4 days or 72 days after transplantation, mice were sacrificed and gels were isolated from the injection site, fixed and stained with either H&E or anti-CD90 antibodies. Each group was consisted of three mice.

As shown in FIG. 20, round cells were observed in gels after H&E staining (H&E) for both 4 days of incubation (Day 4) and 72 days of incubation (Day 72). Furthermore, CD90-positive round cells abundantly observed in gels on Day 4, which were also observed in gels on Day 72. These results suggest that quiescent MSCs transplanted in gels remain quiescent MSCs in vivo for a prolonged period.

### Example 25: Transplanting Quiescent ADSCs in Gels Enhances Wound Healing in Diabetic Mice

A lack of or impaired wound healing is one of the major complications in diabetes, which seriously affects the quality of life or even threats the life of diabetes patients. Enhanced wound healing by MSCs and MSC-derived exosomes have been reported, although MSC-based cell therapies to treat diabetic wound have not been established as yet. Extremely low engulfment rate of MSCs after administrating them into patients, as well as SASP of MSCs induced by a proinflammatory environment due to diabetes, may abolish the beneficial effect of MSCs on wound healing. The data suggest that quiescent MSCs stay in the transplants as quiescent MSCs for a prolonged period, when they are transplanted in gels, which are capable of inducing and maintaining quiescence in MSCs. Furthermore, quiescence makes MSCs resistant to exhibit SASP despite of an oxidative stress, such as high glucose treatment. Thus, the effect of transplanting quiescent ADSCs in gels on wound healing under a diabetic condition was investigated.

Diabetes was induced in C57BL/6 male mice by intraperitoneally injecting streptozotocin (STZ), as described previously. For non-diabetic mice, PBS was injected, instead of STZ. 1 month after STZ injection, wound were created by making a 1.0 cm×1.0 cm incision on the shaved dorsal regions of each mouse as described previously. Right after making an incision, either 300 μl 3D-NANOFIBGROW-I gel, 2.1×10⁶ ADSCs suspended in PBS, or 300 μl 3D-NANOFIBGROW-I gel with 2.1×10⁶ ADSCs was prepared and subcutaneously injected into the back of STZ mice, which was approximately 1.0 cm apart from the skin incision. For non-diabetic mice, 300 μl 3D-NANOFIBGROW-I gel was injected. Each group was consisted of three mice. The size of each wound was measured 3 days (Day 3), 6 days (Day 6) or 10 days (Day 10) after skin incision, which was compared with the size right after each skin incision.

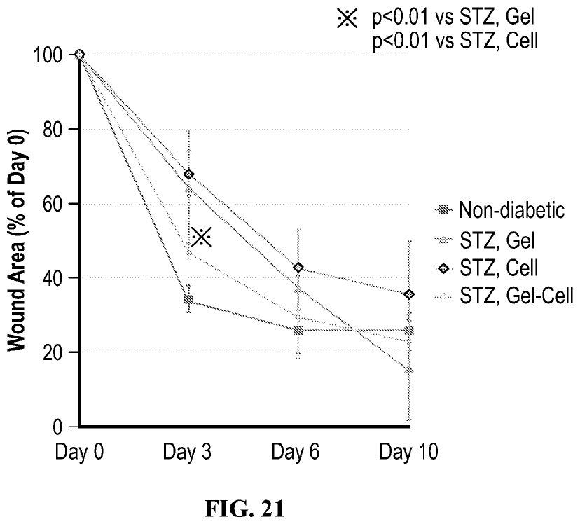

As shown in FIG. 21, non-diabetic mice administrated with gels (Non-diabetic) exhibited a quickest recovery, which was followed by STZ mice administrated with gels containing quiescent ADSCs (STZ, Gel-Cell). STZ mice administrated with either gels only (STZ, Gel) or ADSCs only (STZ, Cell) exhibited a slowest recovery, although all four groups of mice achieved a similar level of recovery on Day 10. On Day 3, the sizes of wounds in STZ mice administrated with gels containing quiescent ADSCs (STZ, Gel-Cell) were significantly smaller than those in STZ mice administrated with either gels only (STZ, Gel) or ADSCs only (STZ, Cell). These results suggest administrating quiescent ADSCs in gels enhances wound healing under diabetic condition.

### Example 26: Quiescence Rejuvenates ADSCs From High Glucose-Induced SASP and Enhances Wound Healing Under Diabetic Environment Similarly to Quiescent ADSCs That Have Not Experienced SASP

High glucose treatment caused SASP in ADSCs, which was restored by introducing and maintaining quiescence in those cells. For instance, once high glucose-treated ADSCs were embedded in 3D-NANOFIBGROW-I gels and become quiescent, they no longer responded to high glucose treatment in their dehydrogenase activity (FIG. 15). Thus, non-quiescent ADSCs may develop SASP, when they are subjected to a diabetic condition, which could be one of the reasons why STZ mice administrated with ADSCs without gels showed a delayed wound healing in FIG. 21. However, it is also reasonable to attribute a delayed wound healing in STZ mice administrated with ADSCs without gels to rapid disappearance of ADSCs after subcutaneous injection, when a sustained survival of quiescent ADSCs in gels in vivo is considered (FIGS. 16 and 20). To establish a contribution of recovering ADSCs from SASP by introducing and maintaining quiescence in those cells to enhance wound healing in diabetic mice, non-quiescent ADSCs were subjected to either normal glucose concentration or high glucose treatment before transplantation. Then quiescence was induced in both groups of ADSCs by embedding them in gels, which were transplanted to diabetic mice to compare the recovery of wound healing between them.

To induce SASP in ADSCs in vitro, ADSCs were cultured in a medium that contained either 5 mM glucose (Normal Glucose) or 35 mM glucose (High Glucose). To investigate the process of wound healing under diabetes, STZ mice were prepared and skin incision was made as described in Example 25. Either 350 μl 3D-NANOFIBGROW-I gel (STZ Gel), or 350 μl 3D-NANOFIBGROW-I gel with 3.2×10⁵ ADSCs cultured under a normal glucose level (ADSC+Gel Normal Glucose) or 3.2×10⁵ ADSCs cultured under a high glucose level (ADSC+Gel High Glucose) was prepared and subcutaneously injected into the back of STZ mice as described in Example 26.

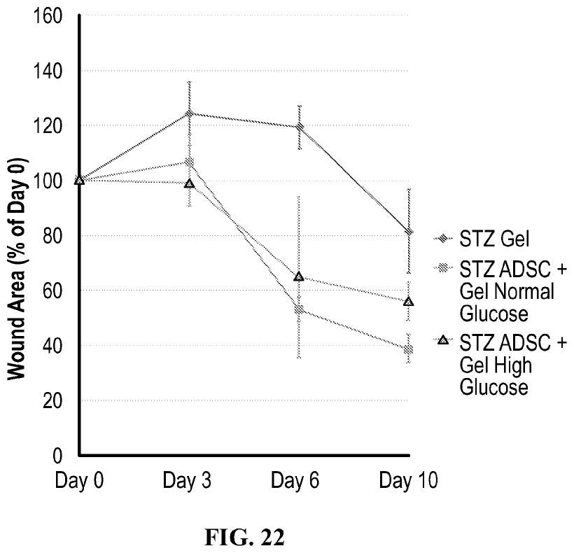

As shown in FIG. 22, transplants containing quiescent ADSCs enhanced wound healing in STZ mice, as those mice exhibited significantly rapid recovery, when compared with STZ mice transplanted with gels that did not have ADSCs in them (STZ Gel) on Day 3 and Day 6. However, there was no statistically significant difference in wound healing throughout the period of this experiment between mice transplanted with gels congaing quiescent ADSCs that have been cultured at a normal glucose level in vitro (ADSC+Gel Normal Glucose) and mice transplanted with gels congaing quiescent ADSCs that have been cultured at a high glucose level in vitro (ADSC+Gel High Glucose). These results suggest that once ADSCs become quiescent, they are able to recover from SASP condition and exert their beneficial effects on wound healing similarly to quiescent ADSCs that have not experienced SASP.

1. A method of reducing oxidative stress in a cell, comprising contacting the cell with a quiescent mesenchymal stem cell (MSC).

2. The method according to claim 1, wherein the MSC is adipose tissue-derived stromal cells (ADSCs).

3. The method according to claim 1, wherein the MSC is bone marrow-derived stem cells (BMSCs).

4. The method according to claim 1, wherein the MSC is derived from bone marrow, dental pulp, umbilical cord, Wharton's jelly, amniotic fluid, placenta, peripheral blood, synovium, synovial fluid, endometrium, skin or muscle.

5. The method according to claim 1, wherein the cell is selected from the group consisting of an endothelial cell, a lung cell, and a skin cell.

6. The method according to claim 1, wherein the cell is selected from the group consisting of Cardiomyocyte, Endothelial cells, Vascular smooth muscle cell, Fibroblast, Myofibroblast, Macrophage, Monocyte, Dendritic cell, Immune cell, Lung epithelial cell, Bronchial epithelial cell, Tubular epithelial cell, Podocyte, Interstitial cell, Mesangial cell, Adipocyte, Myotube, Myocyte, Hepatocyte, Biliary epithelial cell, Pancreatic beta cell, Retinal cell, Neuronal cell, Glial cell, Cancer cell, Keratinocyte, Melanocyte, Gastrointestinal epithelial cell, Colon epithelial cell, Osteoblast, Osteocyte, Osteoclast, Gland cell, and Hematopoietic stem cell and progenitor cell

7. The method according to claim 1, wherein the quiescent MSC is prepared by culturing an MSC on a substrate having a rigidity from 150 Pa to 750 Pa.

8. The method according to claim 1, wherein the quiescent MSC is prepared by culturing an MSC on a substrate having a uniform rigidity from 150 Pa to 750 Pa.

9. The method according to claim 7, wherein the substrate is a gel.

10. The method according to claim 7, wherein the substrate is a 2-dimensional gel.

11. The method according to claim 7, wherein the substrate is a 3-dimensional gel.

12. The method according to claim 7, wherein the gel is coated with collagen, fibronectin or a combination thereof.

13. The method according to claim 7, wherein the gel comprises a gelling agent and an acrylamide-bisacrylamide mixture.

14. The method according to claim 13, wherein the gelling agent is a recombinant fibrin or fibrinogen protein.

15. The method according to claim 14, wherein the concentration of the recombinant fibrin or fibrinogen protein in the substrate is 1-20 mg/ml.

16. The method according to claim 1, wherein the method increases exosomal microRNA secretion of the cell.

17. The method according to claim 1, wherein the MSC is treated with glucose.

18. The method according to claim 1, wherein the MSC is cultured in a medium containing at least 25 mM glucose.

19-50. (canceled)

51. A cosmetic or pharmaceutical composition comprising a quiescent mesenchymal stem cell (MSC) and a substrate that adheres to the quiescent MSC.

52-63. (canceled)
